(19) United States
(12) Patent Application Publication
(10) Pub. No.: US 2023/0174636 A1
(43) Pub. Date: Jun. 8, 2023

(54) COMPOSITIONS AND METHODS FOR TREATING CANCER

(71) Applicant: RUSH UNIVERSITY MEDICAL CENTER, Chicago, IL (US)

(72) Inventor: Kalipada PAHAN, Skokie, IL (US)

(73) Assignee: RUSH UNIVERSITY MEDICAL CENTER, Chicago, IL (US)

(21) Appl. No.: 17/924,551

(22) PCT Filed: May 12, 2021
(86) PCT No.: PCT/US2021/031917

§ 371 Date: Nov. 10, 2022

Related U.S. Application Data
(60) Provisional application No. 62/704,475, filed on May 12, 2020.

(51) Int. Cl. C07K 16/24; A61K 39/395; A61K 38/17; A61P 35/00

(52) U.S. Cl. CPC C07K 16/244; A61K 39/3955; A61K 38/1709; A61P 35/00

(57) ABSTRACT

Methods of treating triple negative breast cancer (TNBC) using antibodies against p40 monomer are described herein. In some aspects, methods of treatment for TNBC include administration of antibodies against p40 monomer or administration of antibodies against p40 monomer in combination with a binding peptide such as TLR2-interacting domain of MyD88 (TIDM) peptide or NEMO-binding domain (NBD) peptide. Methods of treating other cancers using antibodies against p40 monomer in combination with a binding peptide such as TIDM peptide or NBD peptide are described herein.

### RELATED APPLICATIONS

This application claims the benefit of U.S. Provisional Application No. 62/704,475, filed May 12, 2020, which is incorporated by reference herein in its entirety.

### SEQUENCE LISTING

The instant application contains a Sequence Listing which has been submitted electronically in ASCII format and is hereby incorporated by reference in its entirety. The ASCII copy, created on May 11, 2021, is named 42960-338899_Sequence_listing_ST25.txt and is 7 KB in size.

### BACKGROUND

### 1. Technical Field

This disclosure generally relates to methods for treating cancer. One aspect provides a method of treating Triple Negative Breast cancer including administering an antibody against p40 monomer to a subject in need of such treatment. Another aspect provides compositions and methods for treating cancer including administering an antibody against p40 monomer and a binding domain peptide to a subject in need of such treatment.

### 2. Description of the Related Art

Although some therapies are available for other breast cancers, there are very few options for triple negative breast cancer (TNBC), an aggressive form of breast cancer that is negative for estrogen receptor, progesterone receptor and HER2. The therapies used for other breast cancers have not been effective for TNBC. Approximately 30% to 40% of patients with TNBC will have a recurrence of disease within 3 to 10 years of treatment with neoadjuvant therapy and surgery. Most patients with recurrent TNBC disease will die from their breast cancer. Therefore, identification of therapeutic advances specific for TNBC is critical.

### SUMMARY OF THE DISCLOSURE

Methods of treating triple negative breast cancer (TNBC) using antibodies against p40 monomer are described herein. In some aspects, methods of treatment for TNBC include administration of antibodies against p40 monomer or administration of antibodies against p40 monomer in combination with a binding peptide such as TLR2-interacting domain of MyD88 (TIDM) peptide or NEMO-binding domain (NBD) peptide. Methods of treating other cancers using antibodies against p40 monomer in combination with a binding peptide such as TIDM peptide or NBD peptide are described herein.

Other advantages and features will be apparent from the following detailed description when read in conjunction with the attached drawings.

### BRIEF DESCRIPTION OF THE DRAWINGS

FIG. 1A-1D. Levels of IL-12 family of cytokines in serum of breast cancer patients. Serum of drug-naïve breast cancer patients (n=12) and age-matched healthy controls (n=12) obtained from Discovery Life Sciences (Los Osos, Calif.) were analyzed for p40 (1A), p40₂ (1B), IL-12 (1C), and IL-23 (1D) by sandwich ELISA. *p<0.05; ***p<0.001.

FIG. 2A-2F. Monoclonal antibody-mediated neutralization of p40 induces death of different human TNBC cells. Levels of p40 and p40₂ were measured in supernatants of human TNBC cells (2A, BT-549; 2B, HCC70) by ELISA. Results are mean±SD of three different experiments. *p<0.05; **p<0.01. BT-549 (2C & 2E) and HCC70 (2D & 2F) cells were treated with p40 mAb for 24 h under serum-free condition followed by monitoring MTT metabolism (2C-2D) and LDH release (2E-2F). Results are mean±SD of three separate experiments. *p<0.05; **p<0.01; ***p<0.001.

FIG. 3A-3D. Regression of TNBC tumor in vivo in patient-derived xenograft (PDX) mice by p40 mAb. 3A) Female 6-8 week old NOD scid gamma (NSG) mice were engrafted TNBC tumor fragments at passage P1-P9 (invasive ductal carcinoma; TNBC ER^{-/-}/PR^{-/-}/HER2^{-/-}) in the flank. After 6 weeks of engraftment, levels of p40 and p40₂ were measured in serum by sandwich ELISA. Results are mean±SEM of four mice (n=4) per group. **p<0.01. 3B) After about 4 weeks of engraftment, when tumors of PDX mice (n=5 per group) were 0.6-0.8 cm² in size, mice were treated with p40 mAb (right panel) and hamster IgG (middle panel) at a dose of 2 mg/kg body wt once a week. After 2 weeks, tumors were labeled with Alexa800 conjugated 2DG dye via tail vein injection and then imaged in Licor Odyssey infrared imaging system. Results were compared with control group (Left panel). 3C) Tumors were excised from the flank of all groups of mice. Five mice (n=5) were included in each group. 3D) Tumor size was monitored every alternate day. Results are mean±SEM of five different mice.

FIG. 4A-4H. Stimulation of death response in TNBC tumor of PDX mice by p40 mAb. Female 6-8 week old NSG mice were engrafted TNBC tumor in the flank. After about 4 weeks of engraftment, when tumors of PDX mice (n=5 per group) were 0.6-0.8 cm² in size, mice were treated with p40 mAb and hamster IgG at a dose of 2 mg/kg body wt once a week. After 2 weeks of treatment, H&E staining (4A) was performed on tumor sections. Tumor tissues were analyzed for the expression of different death-related genes by a custom mRNA array (4B), which was then plotted with heat map explorer software. 4C) Real-time mRNA analyses of 11 different death-related genes. Results are mean±SEM of five mice. ***p<0.001. Tumor tissue sections were double-labeled for actin and TUNEL (4D) followed by counting of TUNEL-positive cells in two sections of each of five mice per group (4E). Single-cell suspensions isolated from tumor tissues were studied by dual FACS for propidium iodide (PI) and Annexin V (4F) in the LSRFortessa analyzer (BD Biosciences) followed by analysis using the FlowJo Software (v10). Quantification of apoptotic (PI⁻Annexin V⁺ early apoptotic+Pl⁺ Annexin V⁺ late apoptotic; 4G) and necrotic (Annexin V⁻PI⁺, 4H) cells. Results are mean±SEM of three PDX mice per group. **p<0.01; ***p<0.001.

FIG. 5A-5H. Stimulation of adaptive immune response in spleen of PDX mice by p40 mAb treatment. Female 6-8 week old NSG mice were engrafted TNBC tumor in the flank. After about 4 weeks of engraftment, when tumors of PDX mice were 0.6-0.8 cm² in size, mice were treated with p40 mAb and hamster IgG at a dose of 2 mg/kg body wt once a week. After 2 weeks of treatment, spleens were harvested and levels of CD4⁺CD8⁺ (5A), CD4⁺IFNγ⁺ (5B) and CD8⁺IFNγ⁺ (5C) T cells were monitored in splenocytes by FACS using a BD LSRFortessa™ cell analyzer. Percentages of CD4⁺CD8⁺ (5D), CD4⁺IFNγ⁺ (5E) and CD8⁺IFNγ⁺ (5F) T cells were calculated. Results are mean±SEM of five mice (n=5) per group (one analysis per mouse). Levels of IFNγ (5G) and IL-10 (5H) were measured in serum of all groups (n=5) of mice by sandwich ELISA. *p<0.05; ***p<0.001.

FIG. 6A-6H. Neutralization of p40 by p40 mAb stimulates adaptive immune response in TNBC tumor of PDX mice. Female 6-8 week old NSG mice were engrafted TNBC tumor in the flank. After about 4 weeks of engraftment, when tumors of PDX mice were 0.6-0.8 cm² in size, mice were treated with p40 mAb and hamster IgG at a dose of 2 mg/kg body wt once a week. After 2 weeks of treatment, tumor tissues were harvested and levels of CD4⁺CD8⁺ (6A), CD4⁺IFNγ⁺ (6B) and CD8⁺IFNγ⁺ (6C) T cells were monitored in single cell suspensions by FACS using a BD LSRFortessa™ cell analyzer. Percentages of CD4⁺CD8⁺ (6D), CD4⁺IFNγ⁺ (6E) and CD8⁺IFNγ⁺ (6F) T cells were calculated. Results are mean±SEM of five mice (n=5) per group (one analysis per mouse). Levels of IFNγ (6G) and IL-10 (6H) were measured in TNBC homogenates of all groups (n=5) of mice by sandwich ELISA. *p<0.05; **p<0.01; ***p<0.001.

FIG. 7A-7C. Neutralization of p40 by p40 mAb induces the infiltration of CD8⁺IFNγ⁺ T cells into TNBC tumor of PDX mice. Female 6-8 week old NSG mice were engrafted TNBC tumor in the flank. After about 4 weeks of engraftment, when tumors of PDX mice were 0.6-0.8 cm² in size, mice were treated with p40 mAb and hamster IgG at a dose of 2 mg/kg body wt once a week. After 2 weeks of treatment, tumor sections were double-labeled for CD8 and IFNγ (7A) followed by counting of CD8⁺ (7B) and IFNγ⁺ (7C) cells in two sections of each of five mice per group. ***p<0.001.

FIG. 8A-8B. Neutralization of p40 by p40 mAb downregulates tumor-associated M2 (TAM2) macrophages, while upregulating TAM1 macrophages in TNBC tumor of PDX mice. Female 6-8 week old NSG mice were engrafted TNBC tumor in the flank. After about 4 weeks of engraftment, when tumors of PDX mice were 0.6-0.8 cm² in size, mice were treated with p40 mAb and hamster IgG at a dose of 2 mg/kg body wt once a week. After 2 weeks of treatment, tumor cross sections were double-immunolabeled for either Iba1 & iNOS or Iba1 & Arg1. DAPI was used to stain nuclei. Cells positive for Iba1 & iNOS (A) and Iba1 & Arg1 (B) were counted in one section (2-3 images per section) of each of five different mice per group. ***p<0.001.

FIG. 9A-9B. Neutralization of p40 by p40 mAb suppresses PD-1/PD-L1 signaling in TNBC tumor of PDX mice. Female 6-8 week old NSG mice were engrafted TNBC tumor in the flank. After about 4 weeks of engraftment, when tumors of PDX mice were 0.6-0.8 cm² in size, mice were treated with p40 mAb and hamster IgG at a dose of 2 mg/kg body wt once a week. After 2 weeks of treatment, tumor cross sections were immunostained for either PD-1 or PD-L1. DAPI was used to stain nuclei. Cells positive for PD-1 (A) and PD-L1 (B) were counted in one section (2-3 images per section) of each of five different mice per group. ***p<0.001.

FIG. 10A-10B. Neutralization of p40 by p40 mAb is not toxic for PDX mice. Female 6-8 week old NSG mice were engrafted TNBC tumor in the flank. After about 4 weeks of engraftment, when tumors of PDX mice were 0.6-0.8 cm² in size, mice were treated with p40 mAb and hamster IgG at a dose of 2 mg/kg body wt once a week. After 2 weeks of treatment, the level of LDH (10A) and ALT (10B) was assayed in serum using assay kits (Sigma). Results are mean±SEM of five mice per group. ***p<0.001.

FIG. 11. The level of p40 monomer is higher than p40 homodimer in human triple negative breast cancer (TNBC) cells. Human TNBC cells (BT549 and HCC-70) were purchased from ATCC and levels of p40 monomer and homodimer were measured in supernatants by sandwich ELISA. Results are mean±S.D. of three separate experiments.

FIG. 12A-12B. The p40 mAb treatment does not alter T-helper 17 (Th17) immune response in a mouse model of TNBC. 12A) TNBC mice (PDX model ID # TM00096) were obtained from Jackson Lab. After 5 weeks of tumor engraftment, when tumors were about 0.5 cm² in size, mice were treated with p40 mAb and hamster IgG at a dose of 2 mg/kg body wt once a week. After 3 weeks of treatment, spleens were harvested and levels of Th17 or CD4⁺IL-17⁺ T cells (12A) were monitored in splenocytes by FACS using a BD LSRFortessa™ cell analyzer. 12B) Percentage of CD4⁺IL-17⁺ T cells were calculated. Results are mean±SEM of three mice (n=3) per group (one analysis per mouse). NS, not significant.

FIG. 13A-13C. Neutralization of p40 monomer by mAb a3-7g induces death of different human cancer cells. LnCAP (13A), Hep3B (13B) and HCC70 (13C) cells at 70-80% confluence were treated with neutralizing mAb a3-7g against p40 for 48 h under serum-free condition followed by monitoring cell viability by MTT assay. Results are mean±S.D. of three separate experiments. **p<0.01 & ***p<0.001 vs. control.

FIG. 14. NEMO-binding domain (NBD) peptide, selective inhibitor of classical NF-kB activation. (SEQ ID NO: 7; SEQ ID NO: 9)

FIG. 15A-15C. The wild type NBD (wtNBD), but not mutated NBD (mNBD), peptide potentiates the efficacy of mAb a3-3a in killing different cancer cells. MCF-7 (15A), Hep3B (15B) and BT-549 (15C) cells plated at 70-80% confluence were treated with neutralizing mAb a3-3a against p40 in the presence or absence of wtNBD and mNBD peptide for 48 h under serum-free condition followed by monitoring cell viability by MTT. Results are mean+S.D. of three separate experiments. *p<0.05, **p<0.01 & ***p<0.001 vs. control.

FIG. 16A-16B. Intranasal administration of wild type NF-κB essential modifier (NEMO)-binding domain (NBD) peptide led to greater regression of tumor in p40 mAb-treated TNBC mice. TNBC mice (PDX model ID # TM00096) were obtained from Jackson Lab. In this model, female NOD Scid gamma (NSG) mice were engrafted TNBC tumor (invasive ductal carcinoma) in the flank. After 5 weeks, when tumors were about 0.5 cm² in size, mice were treated with p40 mAb (a3-3a) at a dose of 2 mg/kg body wt once a week in the presence or absence of daily intranasal treatment of wtNBD and mNBD peptides (0.1 mg/kg body wt/d). 16A) After 3 weeks, tumors were labeled with Alexa800 conjugated 2DG dye via tail vein injection and then imaged in Licor Odyssey infrared imaging system. 16B) Tumor size was monitored after three weeks. Results are mean±SD of five different mice per group.

FIG. 17. TLR2-interacting domain of MyD88 (TIDM) peptide, selective inhibitor of induced TLR2 activation

FIG. 18A-18C. The wild type TIDM (wtTIDM), but not mutated TIDM (mTIDM), peptide potentiates the efficacy of mAb a3-3a in killing different cancer cells. MCF-7 (18A), Hep3B (18B) and BT-549 (18C) cells plated at 70-80% confluence were treated with neutralizing mAb a3-3a against p40 in the presence or absence of wtTIDM and mTIDM peptide for 48 h under serum-free condition followed by monitoring cell viability by MTT. Results are mean±S.D. of three separate experiments. *p<0.05, **p<0.01 & ***p<0.001 vs. control.

FIG. 19A-19B. Intranasal administration of wild type TLR2-interacting domain of MyD88 (wtTIDM) peptide led to greater regression of tumor in p40 mAb-treated TNBC mice. TNBC mice (PDX model ID # TM00096) were obtained from Jackson Lab. In this model, female NOD Scid gamma (NSG) mice were engrafted TNBC tumor (invasive ductal carcinoma) in the flank. After 5 weeks, when tumors were about 0.5 cm² in size, mice were treated with p40 mAb (a3-3a) at a dose of 2 mg/kg body wt once a week in the presence or absence of daily intranasal treatment of wtTIDM and mTIDM peptides (0.1 mg/kg body wt/d). 19A) After 3 weeks, tumors were labeled with Alexa800 conjugated 2DG dye via tail vein injection and then imaged in Licor Odyssey infrared imaging system. 19B) Tumor size was monitored after three weeks. Results are mean±SD of five different mice per group.

FIG. 20A-20B. Selective inhibition of TLR2 by wtTIDM (TLR2-interacting domain of MyD88) peptide and selective inhibition of NF-kB activation by wtNBD (NEMO-binding domain) peptide further stimulate T-cytotoxic 1 (Tc1) immune response in p40 mAb-treated TNBC mice. A) TNBC mice (PDX model ID # TM00096) were obtained from Jackson Lab. After 5 weeks of tumor engraftment, when tumors were about 0.5 cm² in size, mice were treated with p40 mAb and hamster IgG at a dose of 2 mg/kg body wt once a week. In separate groups, mice receiving p40 mAb were also treated with wtTIDM 0 and wtNBD ( ) peptides intranasally. After 3 weeks of treatment, spleens were harvested and levels of Tc1 or CD8⁺IFNγ⁺ T cells (20A) were monitored in splenocytes by FACS using a BD LSRFortessa™ cell analyzer. 20B) Percentage of CD8⁺IFNγ⁺ T cells were calculated. Results are mean±SEM of three mice (n=3) per group (one analysis per mouse). **p<0.01 vs p40 mAb.

FIG. 21. Intranasal wtTIDM peptide stimulates the p40 mAb-mediated regression of TNBC tumor in vivo in patient-derived xenograft (PDX) mice. Female 6-8 week old NOD scid gamma (NSG) mice were engrafted TNBC tumor fragments at passage P1-P9 (invasive ductal carcinoma; TNBC ER^{-/-}/PR^{-/-}/HER2^{-/-}) in the flank. After about 4 weeks of engraftment, when tumors of PDX mice were 0.6-0.8 cm² in size, mice were treated with p40 mAb and hamster IgG at a dose of 2 mg/kg body wt once a week. Mice were also treated with wild type (wt) and mutated (m) TLR2-interacting domain of MyD88 (TIDM) peptide at a dose of 0.1 mg/kg body wt daily via intranasal route. After 2 weeks, tumors were labeled with Alexa800 conjugated 2DG dye via tail vein injection and then imaged in Licor Odyssey infrared imaging system.

FIG. 22. Intranasal wtTIDM peptide stimulates the p40 mAb-mediated regression of TNBC tumor in vivo in patient-derived xenograft (PDX) mice. Female 6-8 week old NOD scid gamma (NSG) mice were engrafted TNBC tumor fragments at passage P1-P9 (invasive ductal carcinoma; TNBC ER^{-/-}/PR^{-/-}/HER2^{-/-}) in the flank. After about 4 weeks of engraftment, when tumors of PDX mice were 0.6-0.8 cm² in size, mice were treated with p40 mAb and hamster IgG at a dose of 2 mg/kg body wt once a week. Mice were also treated with wild type (wt) and mutated (m) TLR2-interacting domain of MyD88 (TIDM) peptide at a dose of 0.1 mg/kg body wt daily via intranasal route. After 2 weeks of treatment, H&E staining was performed on tumor sections. Results represent analysis of five different mice per group.

FIG. 23A-23B. Intranasal wtTIDM peptide stimulates the p40 mAb-mediated induction of IFNγ while reducing the level of IL-10 in TNBC tumor tissues of patient-derived xenograft (PDX) mice. Female 6-8 week old NOD scid gamma (NSG) mice were engrafted TNBC tumor fragments at passage P1-P9 (invasive ductal carcinoma; TNBC ER^{-/-}/PR^{-/-}/HER2^{-/-}) in the flank. After about 4 weeks of engraftment, when tumors of PDX mice were 0.6-0.8 cm² in size, mice were treated with p40 mAb and hamster IgG at a dose of 2 mg/kg body wt once a week. Mice were also treated with wild type (wt) and mutated (m) TLR2-interacting domain of MyD88 (TIDM) peptide at a dose of 0.1 mg/kg body wt daily via intranasal route. After 2 weeks of treatment, levels of human IFNγ (23A) and human IL-10 (23B) were measured in tumor tissue extracts by sandwich ELISA (Thermofisher). Results are mean±SEM of 6-7 mice per group. ***p<0.001.

FIG. 24A-24B. Intranasal wtTIDM peptide stimulates the p40 mAb-mediated induction of IFNγ while reducing the level of IL-10 in serum of patient-derived xenograft (PDX) mouse model of TNBC. Female 6-8 week old NOD scid gamma (NSG) mice were engrafted TNBC tumor fragments at passage P1-P9 (invasive ductal carcinoma; TNBC ER^{-/-}/PR^{-/-}/HER2^{-/-}) in the flank. After about 4 weeks of engraftment, when tumors of PDX mice were 0.6-0.8 cm² in size, mice were treated with p40 mAb and hamster IgG at a dose of 2 mg/kg body wt once a week. Mice were also treated with wild type (wt) and mutated (m) TLR2-interacting domain of MyD88 (TIDM) peptide at a dose of 0.1 mg/kg body wt daily via intranasal route. After 2 weeks of treatment, levels of human IFNγ (24A) and human IL-10 (24B) were measured in serum by sandwich ELISA (Thermofisher). Results are mean±SEM of 6-7 mice per group. ***p<0.001.

### DETAILED DESCRIPTION

IL-12 family of cytokines has four different members including p40 monomer (p40), p40 homodimer (p40₂), IL-12 (p40:p35), and IL-23 (p40:p19) [4-9]. Recently we have delineated that p40 is different from other IL-12 family members to selectively inhibit IL-12Rβ1 internalization and suppress autoimmune demyelination [5]. We have also seen that certain cancer cells stave off their death with the help from p40 and that selective depletion of p40 by a functional blocking mAb leads to regression of prostate tumor in mice [4]. Here, we demonstrated that the level of p40 was significantly higher in serum of breast cancer patients than that of age-matched healthy controls. Accordingly, human TNBC cells also produced higher levels of p40 than p40₂ and neutralization of p40 by p40 mAb induced death of TNBC cells. Finally, we delineated that p40 mAb treatment upregulated cytotoxic T cells, stimulated M1 macrophages and suppressed the PD-1/PD-L1 axis, leading to the suppression of TNBC tumor growth in a PDX mouse model. These studies identify an anti-TNBC property of p40 mAb, which may be beneficial for TNBC patients.

Methods of treating triple negative breast cancer (TNBC) using antibodies against p40 monomer are described herein. In some embodiments, antibodies a3-3a and a3-7g against p40 monomer are used. In some aspects, methods of treatment may for TNBC include administration of antibodies against p40 monomer or administration of antibodies against p40 monomer in combination with a binding peptide such as TLR2-interacting domain of MyD88 (TIDM) peptide or NEMO-binding domain (NBD) peptide. Methods of treating other cancers (e.g. prostate cancer, non-TNBC types of breast cancer, pancreatic cancer, liver cancer, ovarian cancer, and other cancers that overexpress p40 monomer) using antibodies against p40 monomer in combination with a binding peptide such as TIDM peptide or NBD peptide are described herein.

As used herein, the term “amount” refers to “an amount effective” or “therapeutically effective amount” of a composition, e.g., antibody or peptide, to achieve a beneficial or desired prophylactic or therapeutic result, including clinical results. A “therapeutically effective amount” of a composition may vary according to factors such as the disease state, age, sex, and weight of the individual, and the ability of the antibody or peptide to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). When a therapeutic amount is indicated, the precise amount of the compositions of the present disclosure to be administered may be determined by a physician with consideration of individual differences in age, weight, tumor size, extent of infection or metastasis, and condition of the patient (subject).

An “antibody” refers to a binding agent that is a polypeptide comprising a light chain or heavy chain immunoglobulin variable region which specifically recognizes and binds an epitope of a target antigen, such as a peptide, lipid, polysaccharide, or nucleic acid containing an antigenic determinant, such as those recognized by an immune cell. The term “antibody” includes antigen binding fragments thereof. The term also includes genetically engineered forms such as chimeric antibodies (for example, humanized murine antibodies), hetero-conjugate antibodies (such as, bispecific antibodies) and antigen binding fragments thereof. See also, Pierce Catalog and Handbook, 1994-1995 (Pierce Chemical Co., Rockford, Ill.); Kuby, J., Immunology, 3rd Ed., W. H. Freeman & Co., New York, 1997. The antibody or immunologically active fragment thereof may be a monoclonal antibody or an immunologically active fragment of a monoclonal antibody. In other embodiments, the antibody or immunologically active fragment thereof is a polyclonal, monoclonal, human, humanized, and chimeric antibody; a single chain antibody or an epitope-binding antibody fragment of such an antibody. In another embodiment, the antibody or immunologically active fragment thereof is a humanized antibody or an immunologically active fragment thereof.

Light and heavy chain variable regions contain a “framework” region interrupted by three hypervariable regions, also called “complementarity-determining regions” or “CDRs.” The CDRs may be defined or identified by conventional methods, such as by sequence according to Kabat et al., (Wu, TT and Kabat, E. A., J Exp Med. 132(2):211-50, (1970); Borden, P. and Kabat E. A., PNAS, 84: 2440-2443 (1987); (see, Kabat et al., Sequences of Proteins of Immunological Interest, U.S. Department of Health and Human Services, 1991, which is hereby incorporated by reference), or by structure according to Chothia et al (Choithia, C. and Lesk, A M., J Mal. Biol., 196(4): 901-917 (1987), Choithia, C. et al, Nature, 342: 877-883 (1989)).

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an antibody, Fv, scFv, dsFv, Fab, or other antibody fragment as disclosed herein. References to “V_{L}” or “VL” refer to the variable region of an immunoglobulin light chain, including that of an antibody, Fv, scFv, dsFv, Fab, or other antibody fragment as disclosed herein.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a mouse. In some embodiments, a CAR contemplated herein comprises antigen-specific binding domain that is a chimeric antibody or antigen binding fragment thereof.

In certain embodiments, the antibody is a humanized antibody (such as a humanized monoclonal antibody) that specifically binds to a surface protein on a tumor cell. A “humanized” antibody is an immunoglobulin including a human framework region and one or more CDRs from a non-human (for example a mouse, rat, or synthetic) immunoglobulin. Humanized antibodies may be constructed by means of genetic engineering (see for example, U.S. Pat. No. 5,585,089).

“Camel Ig” or “camelid VHH” as used herein refers to the smallest known antigen binding unit of a heavy chain antibody (Koch-Nolte, et al., FASEB J, 21: 3490-3498 (2007)). A “heavy chain antibody” or a “camelid antibody” refers to an antibody that contains two VH domains and no light chains (Riechmann L. et al., J Immunol. Methods 231:25-38 (1999); WO94/04678; WO94/25591; U.S. Pat. No. 6,005,079).

“IgNAR” of “immunoglobulin new antigen receptor” refers to class of antibodies from the shark immune repertoire that consist of homodimers of one variable new antigen receptor (VNAR) domain and five constant new antigen receptor (CNAR) domains.

Papain digestion of antibodies produces two identical antigen-binding fragments, called “Fab” fragments, each with a single antigen-binding site, and a residual “Fc” fragment, whose name reflects its ability to crystallize readily. The Fab fragment contains the heavy- and light chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” is the minimum antibody fragment which contains a complete antigen-binding site. In a single-chain Fv (scFv) species, one heavy- and one light-chain variable domain may be covalently linked by a flexible peptide linker such that the light and heavy chains may associate in a “dimeric” structure analogous to that in a two-chain Fv species.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., PNAS USA 90: 6444-6448 (1993). Triabodies and tetrabodies are also described in Hudson et al., Nat. Med. 9: 129-134 (2003).

“Single domain antibody” or “sdAb” or “nanobody” refers to an antibody fragment that consists of the variable region of an antibody heavy chain (VH domain) or the variable region of an antibody light chain (VL domain) (Holt, L., et al., Trends in Biotechnology, 21(11): 484-490).

“Single-chain Fv” or “scFv” antibody fragments comprise the VH and VL domains of antibody, wherein these domains are present in a single polypeptide chain and in either orientation (e.g., VL-VH or VH-VL). Generally, the scFv polypeptide further comprises a polypeptide linker between the VH and VL domains which enables the scFv to form the desired structure for antigen binding. For a review of scFv, see, e.g., Pluckthin, in The Pharmacology of Monoclonal Antibodies, vol. 113, Rosenburg and Moore eds., (Springer-Verlag, New York, 1994), pp. 269-315.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

Formulations of therapeutic agents can be prepared by mixing with physiologically acceptable carriers, excipients, or stabilizers in the form of, e.g., lyophilized powders, slurries, aqueous solutions, lotions, or suspensions (see, e.g., Hardman et al., (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, N.Y.; Lieberman, et al. (eds.) (1990) Pharmaceutical Dosage Forms: Tablets, Marcel Dekker, N.Y.; Lieberman, et al. (eds.) (1990) Pharmaceutical Dosage Forms: Disperse Systems, Marcel Dekker, N.Y.; Weiner and Kotkoskie (2000) Excipient Toxicity and Safety, Marcel Dekker, Inc., New York, N.Y.).

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al, (2003) New Engl. J. Med. 348:601-608; Milgrom et al, (1999) New Engl. J. Med. 341: 1966-1973; Slamon et al, (2001) New Engl. J. Med. 344:783-792; Beniaminovitz et al, (2000) New Engl. J. Med. 342:613-619; Ghosh et al, (2003) New Engl. J. Med. 348:24-32; Lipsky et al, (2000) New Engl. J. Med. 343: 1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions as used herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts. Compositions comprising binding agents such as antibodies or fragments thereof can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A specific dose protocol is one involving the maximal dose or dose frequency that avoids significant undesirable side effects. A total weekly dose may be at least 0.05 μg/kg body weight, at least 0.2 μg/kg, at least 0.5 μg/kg, at least 1 μg/kg, at least 10 μg/kg, at least 100 μg/kg, at least 0.2 mg/kg, at least 1.0 mg/kg, at least 2.0 mg/kg, at least 10 mg/kg, at least 25 mg/kg, at least 30 mg/kg, at least 40 mg/kg or at least 50 mg/kg (see, e.g., Yang et al, (2003) New Engl. J. Med. 349:427-434; Herold et al, (2002) New Engl. J. Med. 346: 1692-1698; Liu et al, (1999) J. Neurol. Neurosurg. Psych. 67:451-456; Portielji et al, (2003) Cancer Immunol. Immunother. 52: 133-144). The desired dose of antibodies or fragments thereof is about the same as for an antibody or polypeptide, on a moles/kg body weight basis. The desired plasma concentration of the antibodies or fragments thereof is about, on a moles/kg body weight basis. The dose may be at least 15 μg at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg. The doses administered to a subject may number at least 1, 2, 3, 4, 5, 6, 7, 8, 9, 10, 11, or 12, or more. For antibodies or fragments thereof as used herein, the dosage administered to a patient may be 0.0001 mg/kg to 100 mg/kg of the patient's body weight. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight.

The dosage of the antibodies or fragments thereof of as used herein may be calculated using the patient's weight in kilograms (kg) multiplied by the dose to be administered in mg/kg. The dosage of the antibodies or fragments thereof of the invention may be 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight.

Unit dose of the antibodies or fragments thereof of as used herein may be 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 60 mg, 0.25 mg to 40 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosage of the antibodies or fragments thereof of as used herein the invention may achieve a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in a subject. Alternatively, the dosage of the antibodies or fragments thereof as used herein may achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in the subject.

Doses of antibodies or fragments thereof as used herein may be repeated and the administrations may be separated by at least 1 day, 2 days, 3 days, 5 days, 7 days, 10 days, 15 days, 30 days, 45 days, 2 months, 75 days, 3 months, or at least 6 months.

An effective amount for a particular patient may vary depending on factors such as the condition being treated, the overall health of the patient, the method route and dose of administration and the severity of side effects (see, e.g., Maynard et al., (1996) A Handbook of SOPs for Good Clinical Practice, Interpharm Press, Boca Raton, Fla.; Dent (2001) Good Laboratory and Good Clinical Practice, Urch PubL, London, UK).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., (1983) Biopolymers 22:547-556; Langer et al., (1981) J. Biomed. Mater. Res. 15: 167-277; Langer (1982) Chem. Tech. 12:98-105; Epstein et al, (1985) Proc. Natl. Acad. Sci. USA 82:3688-3692; Hwang et al., (1980) Proc. Natl. Acad. Sci. USA 77:4030-4034; U.S. Pat. Nos. 6,350,466 and 6,316,024). Where necessary, the composition may also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

A composition of the anti-p40 monomer antibody or immunologically active fragment thereof described herein may also be administered via one or more routes of administration using one or more of a variety of methods known in the art. As will be appreciated by the skilled artisan, the route and/or mode of administration will vary depending upon the desired results. Selected routes of administration for antibodies or fragments thereof of the invention include intravenous, intramuscular, intradermal, intraperitoneal, subcutaneous, spinal or other parenteral routes of administration, for example by injection or infusion. Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the invention can be administered via a non-parenteral route, such as a topical, epidermal or mucosal route of administration, for example, intranasally, orally, vaginally, rectally, sublingually or topically. In one embodiment, the antibodies or fragments thereof of the invention is administered by infusion. In another embodiment, the multispecific epitope binding protein of the invention is administered subcutaneously. If the antibodies or fragments thereof of the invention are administered in a controlled release or sustained release system, a pump may be used to achieve controlled or sustained release (see Langer, supra; Sefton, (1987) CRC Crit. Ref Biomed. Eng. 14:20; Buchwald et al., (1980), Surgery 88:507; Saudek et al, (1989) N. Engl. J. Med. 321:574). Polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, (1983) J. Macromol. Sci. Rev. Macromol. Chem. 23:61; see also Levy et al., (1985) Science 228: 190; During et al, (1989) Ann. Neurol. 25:351; Howard et al, (1989) J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer, (1990), Science 249: 1527-1533). Any technique known to one of skill in the art can be used to produce sustained release formulations comprising one or more antibodies or fragments thereof of the invention. See, e.g., U.S. Pat. No. 4,526,938, PCT publication WO 91/05548, PCT publication WO 96/20698, Ning et al, (1996), Radiotherapy & Oncology 39: 179-189, Song et al, (1995) PDA Journal of Pharmaceutical Science & Technology 50:372-397, Cleek et al., (1997) Pro. Intl Symp. Control. Rel. Bioact. Mater. 24:853-854, and Lam et al, (1997) Proc. Intl. Symp. Control Rel. Bioact. Mater. 24:759-760, each of which is incorporated herein by reference in their entirety.

If the antibodies or fragments thereof are administered topically, they can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the compositions comprising antibodies or fragments thereof are administered intranasally, it can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Methods for co-administration or treatment with a second therapeutic agent, e.g., a cytokine, steroid, chemotherapeutic agent, antibiotic, or radiation, are known in the art (see, e.g., Hardman et al., (eds.) (2001) Goodman and Gilman's The Pharmacological Basis of Therapeutics, IO.sup.th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%>; at least 40%>, or at least 50%.

In some aspects, the second therapeutic agent may be a binding peptide comprising TLR2-interacting domain of MyD88 (TIDM) peptide or NEMO-binding domain (NBD) peptide.

Additional therapies (e.g., prophylactic or therapeutic agents), which can be administered in combination with the anti-p40 monomer antibody or immunologically active fragment thereof may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the antibodies or fragments thereof of the invention. The two or more therapies may be administered within one same patient visit.

The anti-p40 monomer antibody or immunologically active fragment thereof and the other therapies may be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the anti-p40 monomer antibody or immunologically active fragment thereof and/or binding peptide can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade, (1989) J. Clin. Pharmacol. 29:685). Exemplary targeting moieties include folate or biotin (see, e.g., U.S. Pat. No. 5,416,016 to Low et al); mannosides (Umezawa et al, (1988) Biochem. Biophys. Res. Commun. 153: 1038); antibodies (Bloeman et al, (1995) FEBS Lett. 357: 140; Owais et al., (1995) Antimicrob. Agents Chemother. 39: 180); surfactant protein A receptor (Briscoe et al, (1995) Am. J. Physiol. 1233: 134); p 120 (Schreier et al, (1994) J. Biol. Chem. 269:9090); see also K. Keinanen; M. L. Laukkanen (1994) FEBS Lett. 346: 123; J. J. Killion; I. J. Fidler (1994) Immunomethods 4:273.

Non-limiting examples of protocols for the administration of pharmaceutical composition comprising anti-p40 monomer antibody or immunologically active fragment thereof alone or in combination with other therapies to a subject in need thereof are provided. The therapies (e.g., prophylactic or therapeutic agents) of the combination therapies can be administered concomitantly or sequentially to a subject. The therapy (e.g., prophylactic or therapeutic agents) of the combination therapies of the present invention can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies can be administered to a subject concurrently. The term “concurrently” is not limited to the administration of therapies (e.g., prophylactic or therapeutic agents) at exactly the same time, but rather it is meant that a pharmaceutical composition comprising antibodies or fragments thereof of the invention are administered to a subject in a sequence and within a time interval such that the antibodies of the invention can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various embodiments, the therapies (e.g., prophylactic or therapeutic agents) are administered to a subject less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other embodiments, two or more therapies (e.g., prophylactic or therapeutic agents) are administered to a within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

The practice of the disclosure will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

All publications, patents and patent applications cited herein are hereby incorporated by reference in their entirety.

p40 Monomer Binding Agents

In some embodiments, the agent that is used in the methods of treatment described herein bind to p40 monomer is an antibody and as used herein includes monoclonal antibody or antibody derivative. Antibody derivatives for treatment, in particular for human treatment, include humanized recombinant antibodies, chimeric recombinant antibodies, Fab, Fab′, F(ab′)2 and F(v) antibody fragments, and monomers or dimers of antibody heavy or light chains or intermixtures thereof. The anti p40 monomer binding agent as used here in does not recognize the p40 portion of IL-12 or IL-23. In some embodiments, the p40 monomer binding agent is derived from monoclonal antibody a3-7g or a3-3a. In some embodiments, the p40 monomer binding agent includes the VH chain CDR1, CDR2, and CDR3 and VL chain CDR1, CDR2, and CDR3 of monoclonal antibodies a3-7g or a3-3a.

Binding Peptides

In some embodiments, the p40 monomer binding agent is administered in combination with a binding peptide. In some embodiments, the p40 monomer binding agent is administered in combination with TLR2-interacting domain of MyD88 (TIDM) peptide or NEMO-binding domain (NBD) peptide. In an embodiment, the TIDM peptide comprises the sequence PGAHQK (SEC) ID NO: 1). In another embodiment, the TIDM peptide includes between 6 and 10 amino acids, including the sequence PGAHQK (SEQ ID NO.: 1). In some embodiments, the TIDM peptide includes fewer than 12, 13, 14 or 15 amino acids. In some embodiments, the TIDM peptide consists of SEQ ID NO: 1. In yet another embodiment, the peptide further includes the Antennapedia homeodomain linked to a peptide comprising a TIDM peptide of SEQ ID NO 1. In some embodiments, the Antennapedia homeodomain peptide of drqikiwfqnrrmkwkk (SEQ ID NO: 2) is used. The Antennapedia homeodomain peptide may be linked to the amino or carboy terminus of the TIDM peptide. In another embodiment, the binding peptide sequence is drqikiwfqnrrmkwkkPGAHQK (SEQ ID NO: 3). A mutated TIDM peptide may be used as a control. For example, the mutated TIDM peptide is PGWHGD (SEQ ID NO: 4) and the mutated TIDM peptide may be coupled to the Antennapedia homeodomain of SEO ID NO: 2. Mutated (m) TIDM; drqikiwfqnrmikwkkPGWHGD (SEQ ID NO.: 5)

In yet other embodiments, the p40 monomer binding agent may be administered in combination with an NBD peptide. In an embodiment, the NBD peptide comprises the sequence LDWSWL (SEQ ID NO: 6). In another embodiment, the NBD peptide includes between 6 and 10 amino adds, including the sequence LDWSWL (SEQ ID NO: 6). In some embodiments, the NBD peptide includes fewer than 12, 13, 14 or 15 amino acids. In some embodiments, the NBD peptide consists of SEC) ID NO: 6. In yet another embodiment, the peptide further includes the Antennapedia homeodomain linked to a peptide comprising a NBD peptide of SEQ ID NO 6. In some embodiments, the Antennapedia homeodomain peptide of drqikiwfqnrrmkwkk (SEQ ID NO: 2) is used. The Antennapedia homeodomain peptide may be linked to the amino or carboy terminus of the NBD peptide. In another embodiment, the binding peptide sequence is drwikiwfqnrrmkwkkLDWSWL (SEQ ID NO: 7). A mutated NBD peptide may be used as a control. For example, the mutated NBD peptide is LDASAL (SEQ ID NO: 8) and the mutated NBC peptide may be coupled to the Antennapedia homeodomain of SEQ ID NO:2. Mutated (m) NBD; drqikiwfqnrrmkwkkLDASAL: (SEQ ID NO: 9).

### EXAMPLES

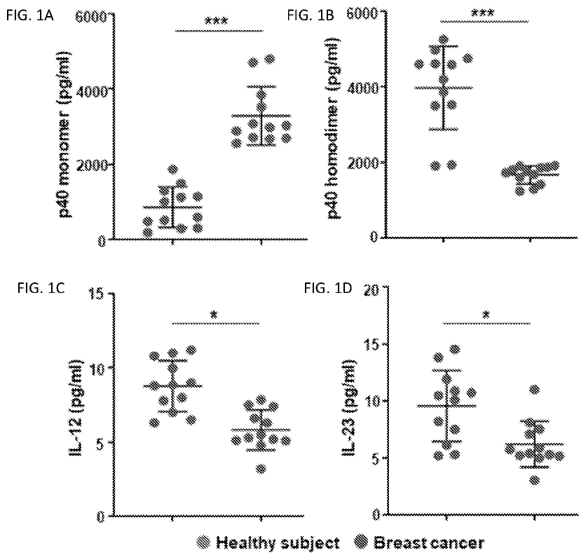

Levels of IL-12, IL-23, p40₂, and p40 in serum of breast cancer patients: Recently we have seen that the level of p40 is much higher in the serum of prostate cancer patients as compared to healthy controls [4]. To understand if this observation is specific to prostate cancer or not, we also measured levels of IL-12, IL-23, p40₂, and p40 in serum of breast cancer patients (n=12) and age-matched healthy controls (n=12). Since disease modifying therapies may alter the levels of these cytokines, we used serum of only pretreated breast cancer patients. Similar to that observed in prostate cancer patients, the level of p40 was significantly higher in serum of breast cancer patients as compared to healthy controls (FIG. 1A). In contrast, levels of p40₂ (FIG. 1B), IL-12 (FIG. 1C) and IL-23 (FIG. 1D) were significantly lower in breast cancer cases relative to healthy controls, indicating the specificity of the finding.

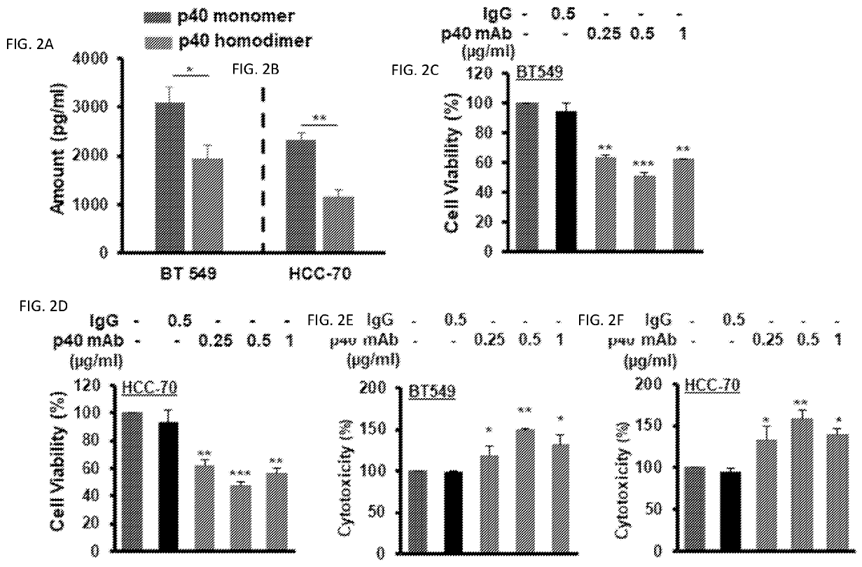

Immunotherapy with monoclonal antibody against IL-12 p40 monomer (p40 mAb) stimulates death in human TNBC cells: Since it was difficult to get serum samples of TNBC cases in sufficient numbers, next, we monitored levels of p40 and p40₂ in human TNBC cell lines. Human TNBC cells (BT-549 and HCC70) were cultured under serum-free condition for 48 h followed by measuring the levels of p40 and p40₂ in supernatants by sandwich ELISA. Similar to that found in serum of breast cancer cases, the level of p40 was significantly higher than p40₂ in supernatants of both BT-549 (FIG. 2A) and HCC70 (FIG. 2B) cells. Therefore, we examined the role of p40 in the survival human TNBC cells using p40 mAb a3-3a. Earlier we demonstrated that p40 mAb a3-3a neutralized the production of nitric oxide and tumor necrosis factor α (TNFα) in peritoneal macrophages induced by only p40, but not p40₂, IL-12 and IL-23 [10]. Recently, we have also seen that single administration of p40 mAb a3-3a, but not control IgG, stimulates clinical symptoms of experimental allergic encephalomyelitis (EAE), an animal model of multiple sclerosis (MS) [5]. This is in sharp contrast to the functions of mAb against p40₂, IL-12 and IL-23 in EAE [11, 18, 19], signifying that function of p40 is different from other IL-12 family members (p40₂, IL-12 and IL-23). Here, we noticed that the p40 mAb a3-3a decreased MTT (FIG. 2C-D) and increased LDH release (FIG. 2E-F) in both BT-549 (FIGS. 2C & E) and HCC70 (FIGS. 2D & F) cells, indicating induction of cell death in TNBC cells by neutralization of p40. This result was specific as control hamster IgG had not effect on either LDH or MTT in human TNBC cells.

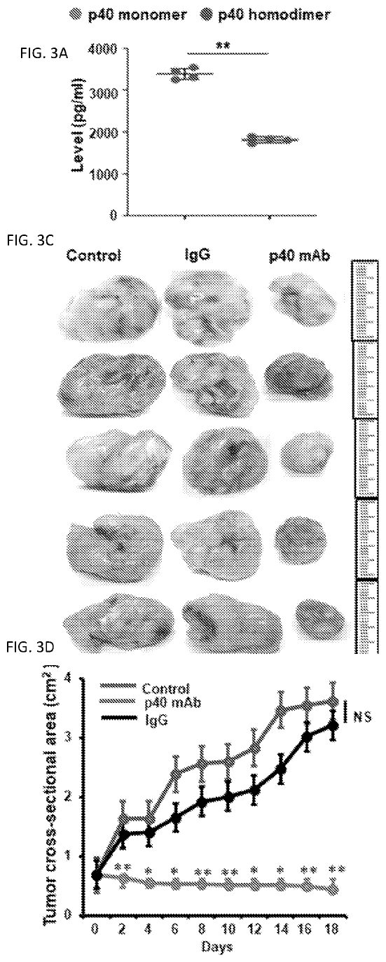

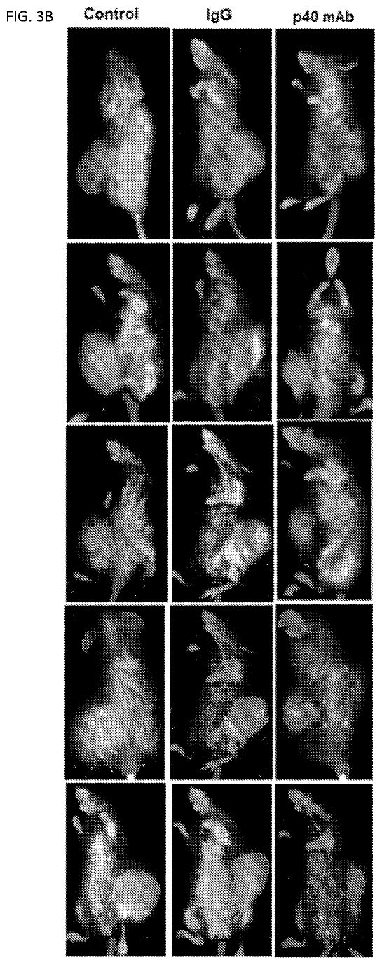

Immunotherapy with p40 mAb induces regression of tumor in patient-derived xenograft (PDX) mouse model of TNBC: In the absence of any effective genetically engineered mouse model for TNBC, PDX models are widely used to evaluate preclinical assessment of any new therapeutic approaches. We used the PDX model (ID # TM00096; Jackson Lab) for this study. In this model, TNBC tumor (invasive ductal carcinoma) were engrafted in the flank of female NOD scid gamma (NSG) mice. At first, we measured the level of p40 in serum of PDX mice and found greater level of p40 than p40₂ in serum about 4 weeks after TNBC tumor engraftment (FIG. 3A). Therefore, to examine the role of p40 in the progression of TNBC tumor, we examined the effect of p40 mAb on tumor size and the death of tumor tissue in PDX mice. When the tumor had reached 0.6-0.8 cm² in area, mice were treated with p40 mAb a3-3a at a dose of 2 mg/kg body/week intraperitoneally (i.p.) for 2 weeks. The tumor size was recorded every alternate day. After 2 weeks of treatment, tumors were labeled with IR dye 800-conjugated 2-deoxy-d-glucose via tail vein injection and then imaged in a LI-COR Odyssey infrared scanner. Interestingly, we observed that administration of p40 mAb significantly reduced the size of tumors as evident from whole-animal IR images (FIG. 3B) and images of excised tumors (FIG. 3C). It was clear from the tumor regression curve that the size of tumors in the p40 mAb-treated group was much less than the control group (FIG. 3D). In contrast, control hamster IgG had no such effect (FIG. 3B-D).

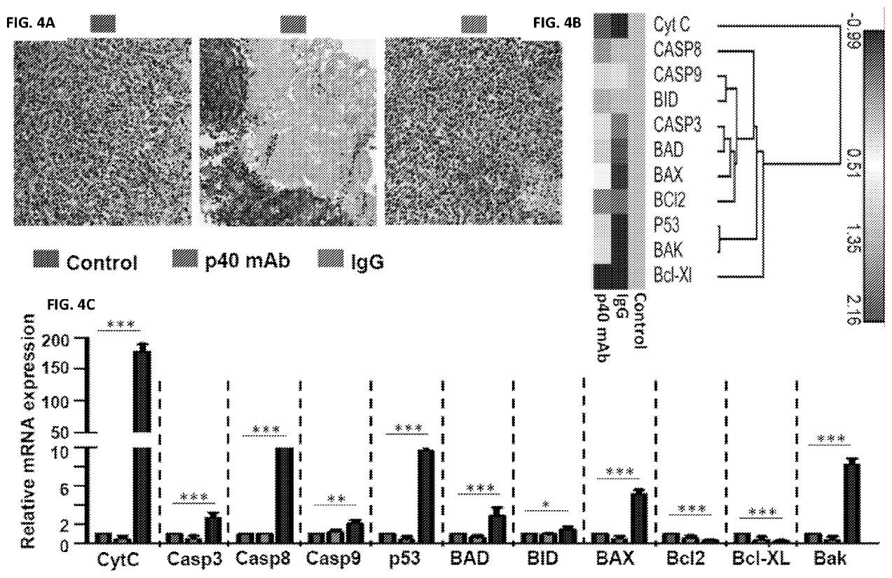

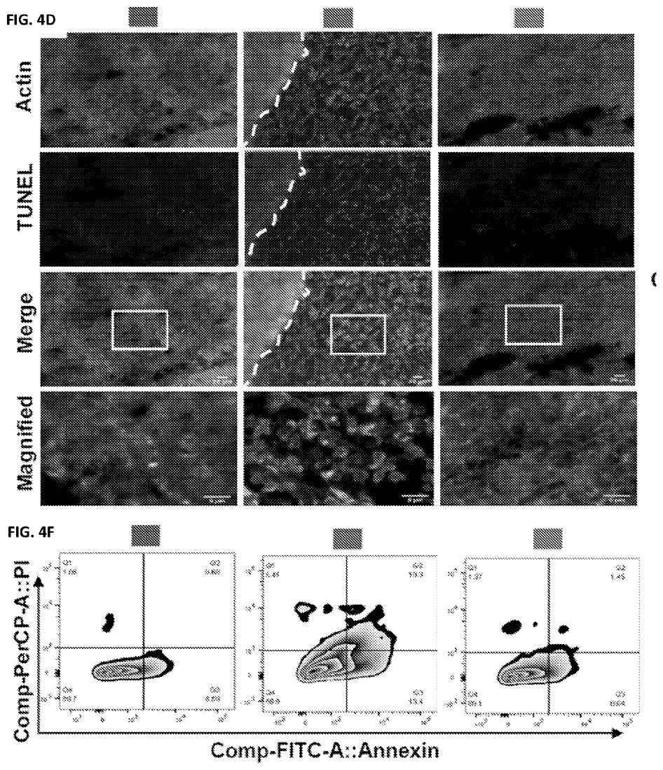

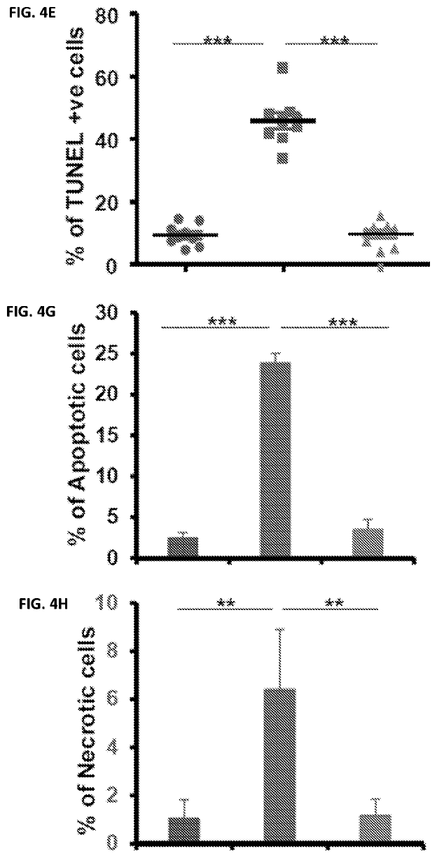

Immunotherapy with p40 mAb stimulates death response in tumor tissues of PDX mouse model of TNBC: Evasion of apoptosis, the cell's natural mechanism for programed cell death, is one of the hallmarks of cancer cells. Therefore, next, we monitored apoptosis or death response in these tumor tissues. Although we did not see complete regression of TNBC tumors by p40 mAb, H&E staining showed almost empty core as we did not find the presence of any live cells in the core of the tumor of p40 mAb-treated PDX mice in comparison to either control untreated or IgG-treated PDX mice (FIG. 4A). To understand the cell death process, we checked the mRNA expression of different genes related to cell death and survival in treated and untreated tumor tissues using a custom gene array. Gene array (FIG. 4B) followed by real-time PCR analysis of individual genes (FIG. 4C) clearly showed that p40 mAb treatment markedly increased the expression of apoptosis-related genes such as cytochrome C, caspase 3, caspase 8, caspase 9, p53, BAD, BID, BAX, and BAK in tumor tissues of PDX mice. On the other hand, we observed decrease in survival-associated genes such as Bcl₂ and Bcl-XL in tumor tissues of p40 mAb-treated PDX mice (FIG. 4B-C). Our TUNEL results also clearly showed that the population of TUNEL-positive cells in the p40 mAb-treated tumors was higher than either control or IgG-treated tumors (FIG. 4D-E). To confirm this observation further, we performed dual FACS analysis with propidium iodide and annexin V (FIG. 4F) and found that p40 mAb treatment markedly increased the level of both apoptotic (FIG. 4G) and necrotic (FIG. 4H) cells in tumor tissues of PDX mice. Together, these results suggest that neutralization of p40 by p40 mAb is capable of inducing cell death in TNBC tumors.

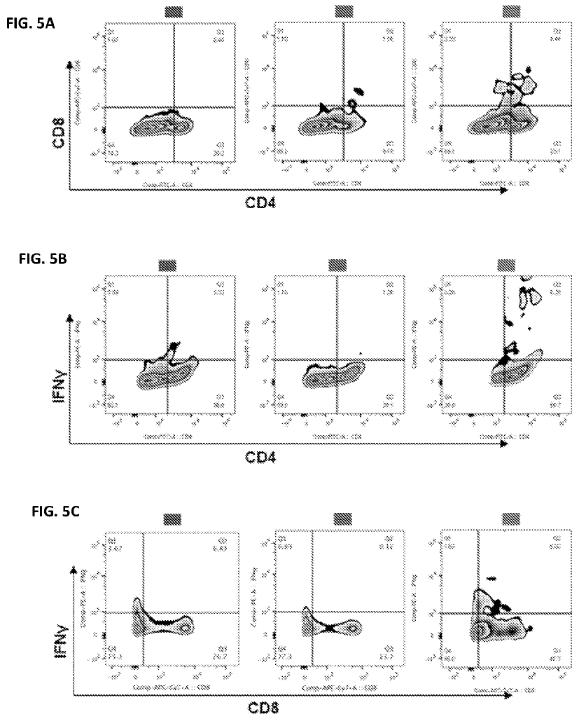

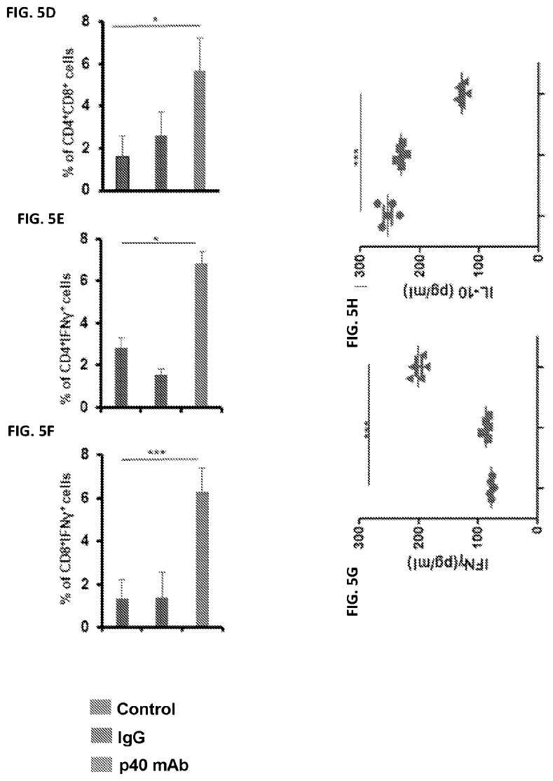

Upregulation of T-helper 1 (Th1) and T-cytotoxic 1 (Tc1) immune responses in vivo in spleen of PDX mice after p40 mAb treatment: Cancer cells do not routinely die off like other cells in the human body as cancer cells are known to escape death due to a change in immune surveillance. Fortunately, we have been endowed with T cytotoxic 1 (Tc1) and T helper 1 (Th1) cells to deal with such a situation. While collaborations between Tc1 and Th1 cells are needed to kill tumor cells [20-22], several studies have reported that both Th1 and Tc1 immune responses are suppressed in cancer patients during disease progression [23]. Although we employed PDX model in NSG mice, a number of studies have demonstrated the development of functional T cells in humanized mouse models [24, 25]. CD3⁺ and CD8⁺ T cells are readily identified in blood, spleen, and bone marrow of a PDX model of TNBC in NSG mice [26]. According to Najima et al [27], human CD8+ T cells in NSG mice also recognize the tumor associated antigen, Wilms tumor 1 (WT1), suggesting that human mature T cells recognizing specific antigens can be generated in the humanized mouse model. Therefore, we monitored the effect of p40 mAb treatment on Th1 and Tc1 responses in treated and untreated PDX mice. While Th1 cells are characterized by CD4 and IFNγ, Tc1 cells are basically CD8⁺IFN⁺. Interestingly, we found that p40 mAb treatment markedly increased the overall adaptive immune response as monitored by an increase in CD4⁺, CD8⁺ as well as CD4⁺CD8⁺ T cells in splenocytes of p40 mAb-treated PDX mice in comparison to either untreated or control IgG-treated PDX mice (FIGS. 5A & D). Furthermore, double labeling of splenocytes for CD4 and IFNγ revealed an upregulation of Th1 response in PDX mice by p40 mAb treatment (FIGS. 5B & E). This result was specific as we did not see any increase in CD4⁺IFNγ⁺Th1 cells by control IgG treatment (FIGS. 5B & E). Similarly, when we labeled splenocytes for CD8 and IFNγ, we found marked increase in CD8⁺IFNγ⁺ Tc1 cells in TNBC mice by treatment with p40 mAb, but not IgG (FIGS. 5C & F). Our ELISA results from serum of PDX mice also demonstrate marked increase in IFNγ, a Th1 and Tc1 cytokine, in serum of PDX mice after treatment with p40 mAb, but not control IgG (FIG. 5G). In contrast, we found significant decrease in IL-10, a Th2 cytokine, in serum of p40 mAb-treated PDX mice (FIG. 5H), highlighting the specificity of our finding that neutralization of p40 by mAb in PDX mice selectively upregulates Th1 and Tc1 responses.

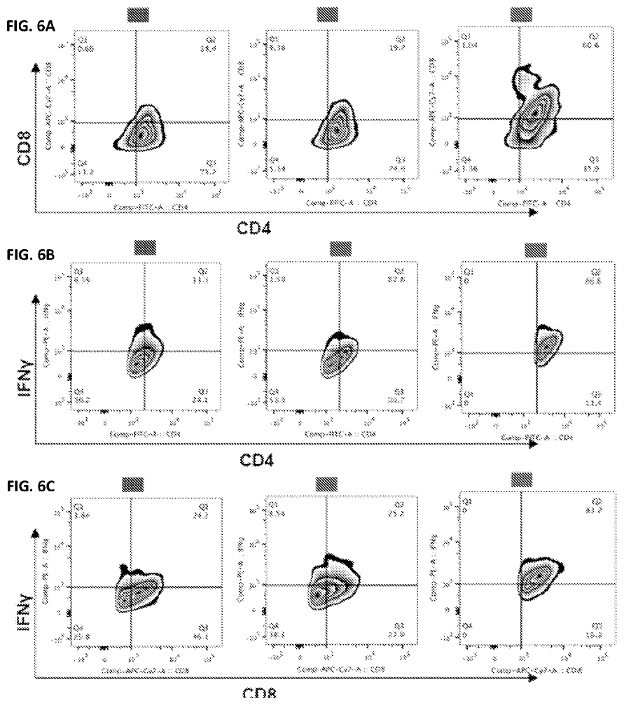

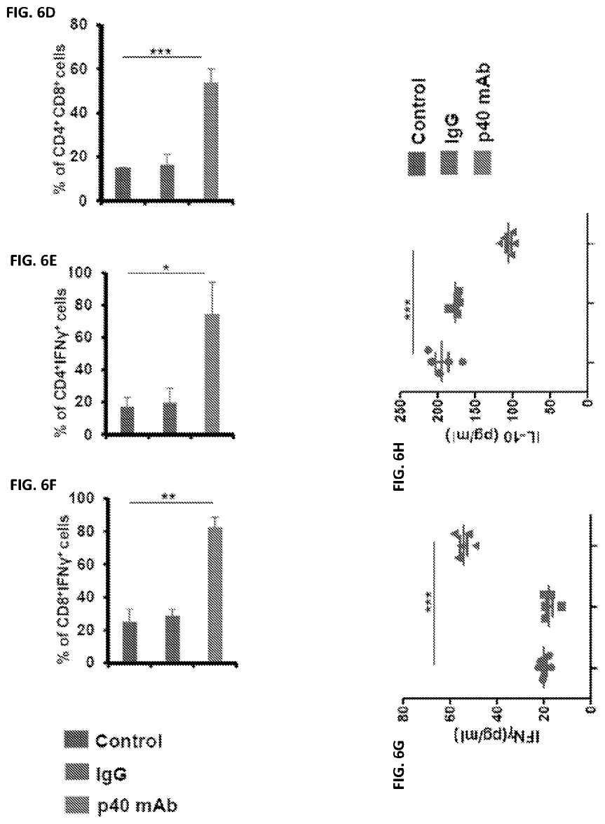

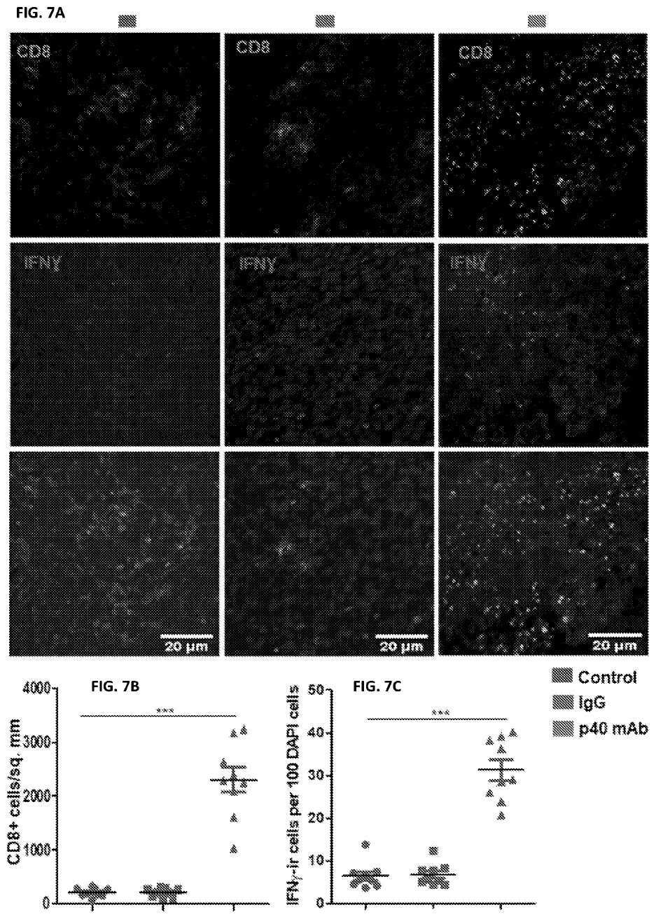

Immunotherapy with p40 mAb increases Th1 and Tc1 immune responses in tumor tissues of PDX mouse model of TNBC: Since p40 mAb upregulated Th1 and Tc1 immune responses in the spleen of PDX mice, next we investigated whether p40 mAb treatment was capable of mounting Th1/Tc1 responses in vivo in tumor tissues. Similar to that observed in spleen, p40 mAb treatment also intensified Th1- and Tc1-driven adaptive immune responses in TNBC tumor as monitored by an increase in CD4⁺CD8⁺ T cells (FIGS. 6A & D), CD4⁺IFNγ⁺ Th1 cells (FIGS. 6B & E) and CD8⁺IFNγ⁺ Tc1 cells (FIGS. 6C & F) in tumor tissues of PDX mice. Since the infiltration of Tc1 cells into the tumor is key to tumor regression, next, we monitored the infiltration of Tc1 cells into TNBC tumor in different groups of PDX mice. Double-labeling of tumor cross sections with antibodies against CD8 and IFNγ (FIG. 7A) followed by counting of CD8+(FIG. 7B) and IFNγ⁺ (FIG. 7C) T cells clearly show marked infiltration of CD8+ cells capable of releasing IFNγ into the tumor of PDX mice after treatment with p40 mAb, but not control IgG.

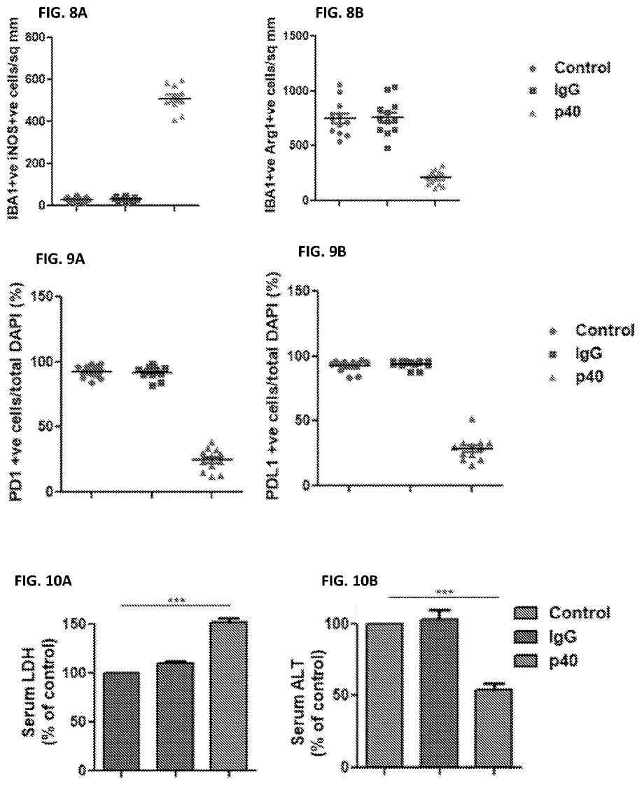

The p40 mAb treatment upregulates Ml macrophages while suppressing M2 ones in the tumor tissues of PDX mouse model of TNBC: Tumor associated macrophages (TAMs) play a critical role in the pathogenesis of various cancers including TNBC [28]. TAMs usually polarize to either M1 exhibiting anti-tumor activity or M2 with tumor promoting functions. While TAM1s express high amounts of inducible nitric oxide synthase (iNOS), TAM2s are characterized by arginase 1 (ARG1) [29]. Therefore, we examined the status of TAM1/TAM2 in TNBC tumor after p40 mAb treatment. As evident from FIG. 8A, the number of iNOS⁺Iba1⁺ TAM1s that was low in control TNBC tumor dramatically increased after treatment with p40 mAb, but not control IgG. In contrast, Arg1⁺Iba1⁺ TAM2s were downregulated in TNBC tumor after treatment with p40 mAb, but not control IgG (FIG. 8B), suggesting that p40 mAb immunotherapy is capable of switching TAM2 to TAM1 in TNBC tumor.

The immunotherapy with p40 mAb downregulates the programmed cell death protein 1 (PD-1)/programmed cell death ligand 1 (PD-L1) axis in tumor tissues of PDX mouse model of TNBC: It has been shown that the PD-1/PD-L1 signaling plays an important role in cancer immune escape [29, 30]. As a result, suppression of the PD-1/PD-L1 axis is associated with a significant clinical response in a wide range of cancer patients. Therefore, here, we examined the effect of p40 mAb immunotherapy on the status of PD-1 and PD-L1. As expected, TNBC tumor tissues readily expressed both PD-1 and PD-L1. However, p40 mAb treatment markedly inhibited the level of both PD-1 (FIG. 9A) and PD-L1 (FIG. 9B), indicating that immunotherapy with p40 mAb blocks the immune escape pathway in TNBC tumor.

The p40 mAb immunotherapy is not toxic in PDX mouse model of TNBC: Alanine aminotransferase (ALT) is probably the most widely used clinical biomarker of liver well-being. Similarly, higher serum LDH than normal levels usually indicates tissue damage. Therefore, to understand whether p40 mAb elicited any toxic effects, we measured LDH and ALT in serum of all groups of mice. While p40 mAb treatment increased the level of serum LDH in PDX mice (FIG. 10A) due to the death of TNBC tumor, the level of serum ALT was markedly inhibited in p40 mAb-treated PDX mice (FIG. 10B), indicating that p40 mAb immunotherapy is not toxic in TNBC mice and that p40 mAb treatment reduces liver toxicity in TNBC mice. These results were specific as control IgG did not alter the level of either LDH or ALT in serum of PDX mice.

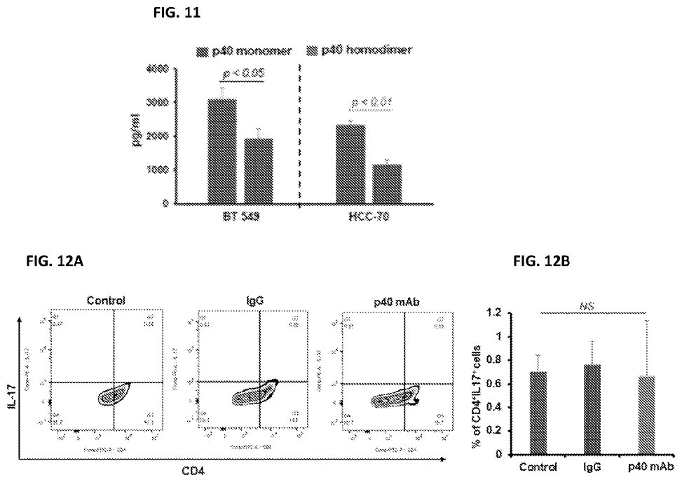

Level of p40 is higher than p40 homodimer (p40₂) in human TNBC cells: First, we monitored the levels of p40 and p40 homodimer (p40₂) in different human TNBC cell lines. BT-549 and HCC-70 cells (ATCC) were cultured under serum-free condition for 48 h, followed by measuring the levels of p40 and p40₂ by sandwich ELISA. The level of p40 was greater than p40₂ in both BT-549 and HCC-70 cells (FIG. 11).

Neutralization of p40 monomer by p40 mAb a3-3a did not control T-helper 17 (Th17) response in TNBC mice: Although Th17 (CD4⁺IL-17⁺) cells participate in the pathogenesis of autoimmune disorders like multiple sclerosis and rheumatoid arthritis, these cells play a controversial role in cancer. While some studies support cancer-destroying ability of Th17 cells, other studies have shown the involvement of Th17 cells in cancer growth. Therefore, we also monitored the effect of p40 mAb treatment on Th17 response in TNBC mice by double-labeling splenocytes for CD4 and IL-17. It was interesting to see that despite upregulating Th1 and Tc1 responses, p40 mAb did not have any significant effect on Th17 cells (FIG. 12A-B). Control IgG treatment also remained unable to modulate Th17 response in TNBC mice (FIG. 12A-B).

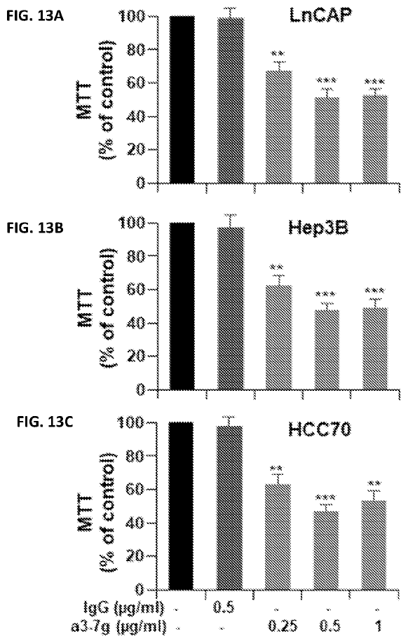

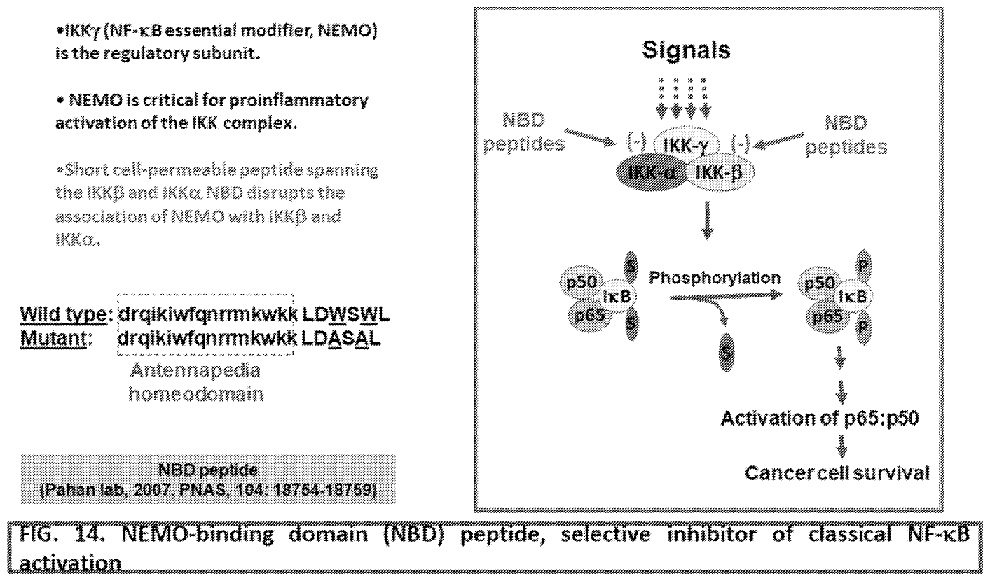

Effect of another neutralizing monoclonal antibodies against p40 monomer (p40 mAb a3-7q) on the survival of human TNBC cells: While the p40 mAb a3-3a is of IgG2a type, the p40 mAb a3-7g is of IgG2b type (Pahan lab, 2008, Hybridoma, 27: 141-151). To understand whether the tumoricidal effect was specific to mAb a3-3a or mAb a3-7g was also capable of inducing death of cancer cells, we analyzed the effect of mAb a3-7g on the survival of different cancer cells. LnCAP (human prostate cancer cells), Hep3B (human liver cancer cells) and HCC-70 (human TNBC cells) were treated with different concentrations of mAb a3-7g for 48 h under serum-free condition followed by monitoring cell survival by MTT assay. As evident from FIG. 13, a3-7g treatment significantly induced the death of LnCAP (FIG. 13A), Hep3B (FIG. 13B) and HCC-70 (FIG. 13C) cells. These results were specific as control IgG remained unable to cause death of these human cancer cells. Moreover, these results suggest that similar to mAb a3-3a, mAb a3-7g is also capable of killing different cancer cells.

Next, we were looking for ways to further increase the cancer destroying effect of p40 mAb.

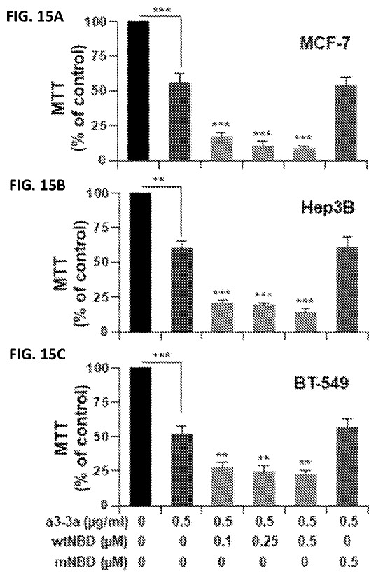

NEMO-binding domain (NBD) peptide increases the efficacy of mAb a3-3a in killing different cancer cells: Low grade inflammation plays a favorable role for the growth of cancer. Therefore, since the activation of NF-kB is crucial for inflammation, we examined if the efficacy of p40 mAb a3-3a could be increased by a specific inhibitor of NF-kB. Others (May et al., 2000, Science, 289: 1550-1554) and we (Pahan lab, 2007, PNAS, 104: 18754-18759) have seen that NF-kB essential modifier (NEMO)-binding domain (NBD) peptide is a specific inhibitor of the activation of NF-kB (FIG. 14). Interestingly, we found that p40 mAb a3-3a induced greater death in MCF-7 (FIG. 15A), Hep3B (FIG. 15B) and BT-549 (FIG. 15C) cells in the presence of wild type NBD (wtNBD) peptide than in its absence. This was not seen in the presence of mutated NBD peptide (FIG. 15), suggesting the specificity of the effect.

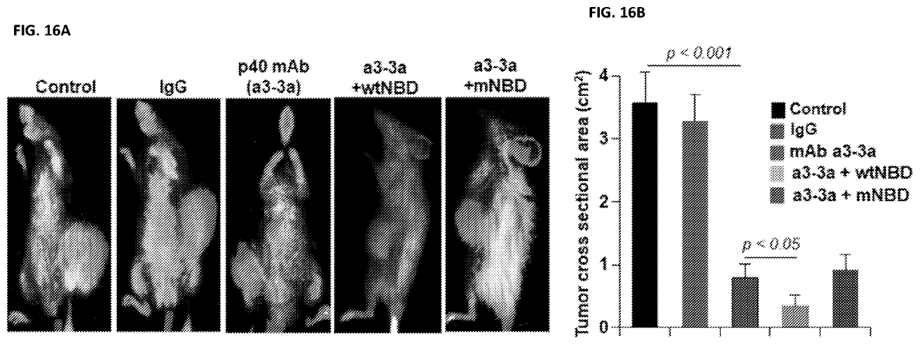

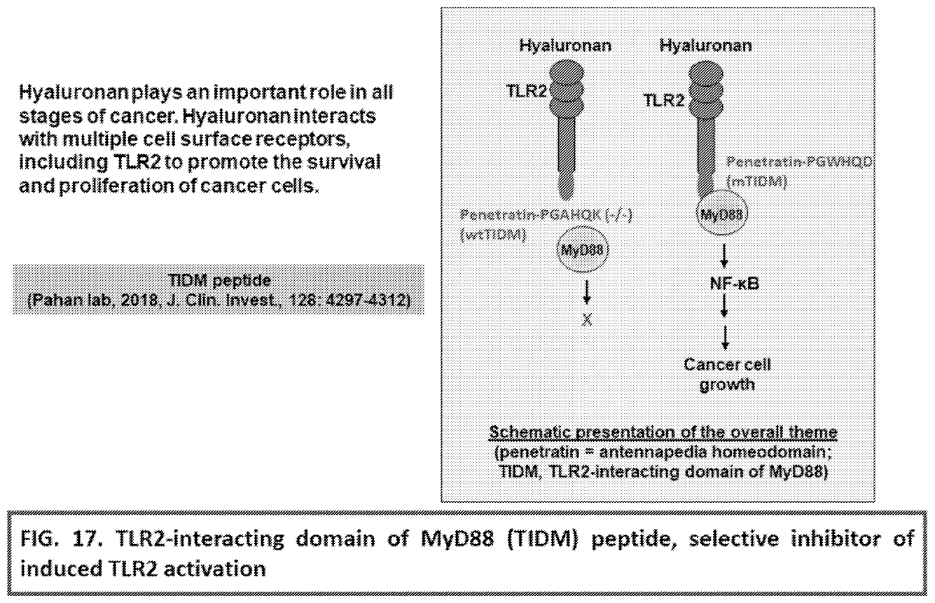

Intranasal administration of wtNBD peptide led to greater regression of tumor in p40 mAb-treated TNBC mice: Since wtNBD peptide exhibited greater death in different human cancer cells, next we examined the effect of wtNBD peptide and p40 mAb a3-3a on the regression of TNBC tumor in mice. While TNBC mice were treated with p40 mAb a3-3a (2 mg/kg body weight) once a week via i.p. injection, same mice were treated with either wtNBD or mNBD peptide (0.1 mg/kg body weight) daily via intranasal route. As evident from FIG. 16, intranasal administration of wtNBD peptide led to greater shrinkage of TNBC tumor in mAb a3-3a-treated TNBC mice. This result was specific as mNBD peptide did not exhibit such effect (FIG. 16).

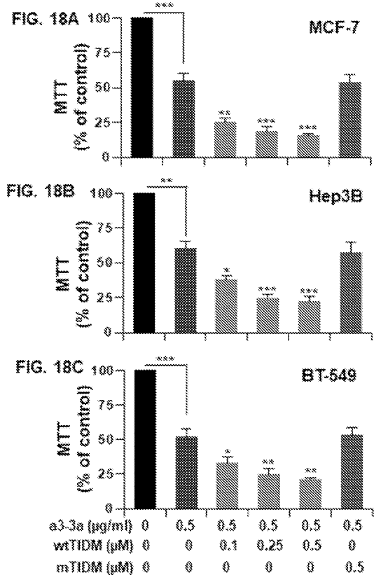

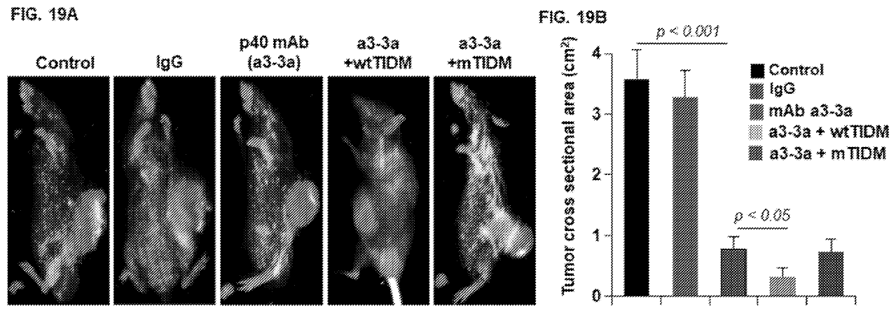

TLR2-interacting domain of MyD88 (TIDM) peptide potentiates the efficacy of mAb a3-3a in killing different cancer cells: Since hyaluronan plays an important role in different stages of cancer and that hyaluronan interacts with multiple cell surface receptors, including TLR2 to promote the survival and proliferation of cancer cells, we decided to target TLR2 in different tumor cells. Recently we have shown selective knockdown of TLR2 (an innate immune component) by TLR2-interacting domain of MyD88 (TIDM) peptide (Pahan lab, 2018, J. Clin. Invest., 128: 4297) (FIG. 17). Therefore, different tumor cells (MCF-7, Hep3B and BT-549) were treated with mAb a3-3a in the presence or absence of wtTIDM and mTIDM peptide for 48 h under serum-free condition. As evident from MTT, the combination of mAb a3-3a and wtTIDM peptide markedly induced death in MCF-7 (FIG. 18A), Hep3B (FIG. 18B) and BT-549 (FIG. 18C) cells.

Intranasal administration of wtTIDM peptide led to greater regression of tumor in p40 mAb-treated TNBC mice: Since wtTIDM peptide induced superior death in different human cancer cells, next we examined if wtTIDM peptide caused greater regression of tumor in TNBC mice in the presence of p40 mAb a3-3a. In this case as well, TNBC mice were treated with p40 mAb a3-3a (2 mg/kg body wt) once a week via i.p. injection wtNBD/mNBD peptide (0.1 mg/kg body wt) daily via intranasal route. After three weeks of treatment, tumors were labeled with Alexa800 conjugated 2DG dye via tail vein injection followed by infrared imaging in Licor Odyssey. Similar to wtNBD peptide, intranasal administration of wtTIDM, but not mTIDM, peptide also caused greater tumor shrinkage in mAb a3-3a-treated TNBC mice (FIG. 19A-B). These results suggest that the combination of mAb a3-3a and intranasal wtTIDM peptide may be an effective strategy to control TNBC.

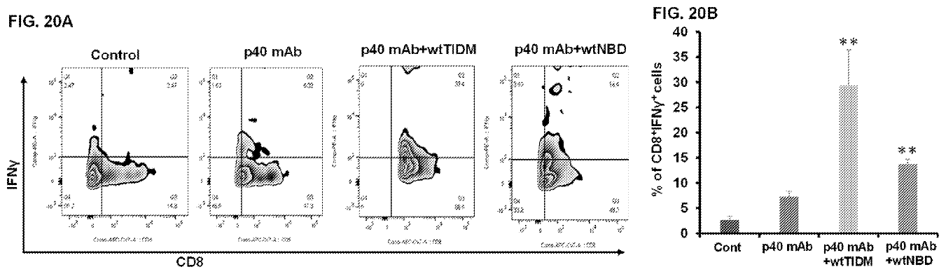

Intranasal administration of either wtNBD or wtTIDM peptide stimulated the cancer-destroying Tc1 response in p40 mAb-treated TNBC mice: Since intranasal wtNBD and wtTIDM peptide led to better tumor regression in mAb a3-3a-treated TNBC mice, next, we investigated underlying mechanism. Driving Tc1 response is always beneficial for cancer. Therefore, we examined if wtTIDM or wtNBD peptide was capable of mounting Tc1 response in a3-3a-treated TNBC mice. Interestingly, we found that intranasal administration of low-dose wtTIDM peptide or wtNBD peptide markedly stimulated the Tc1 response in p40 mAb-treated TNBC mice (FIG. 20), suggesting that either wtTIDM or wtNBD peptide caused greater tumor shrinkage in p40 mAb-treated mice probably via increased Tc1 response.

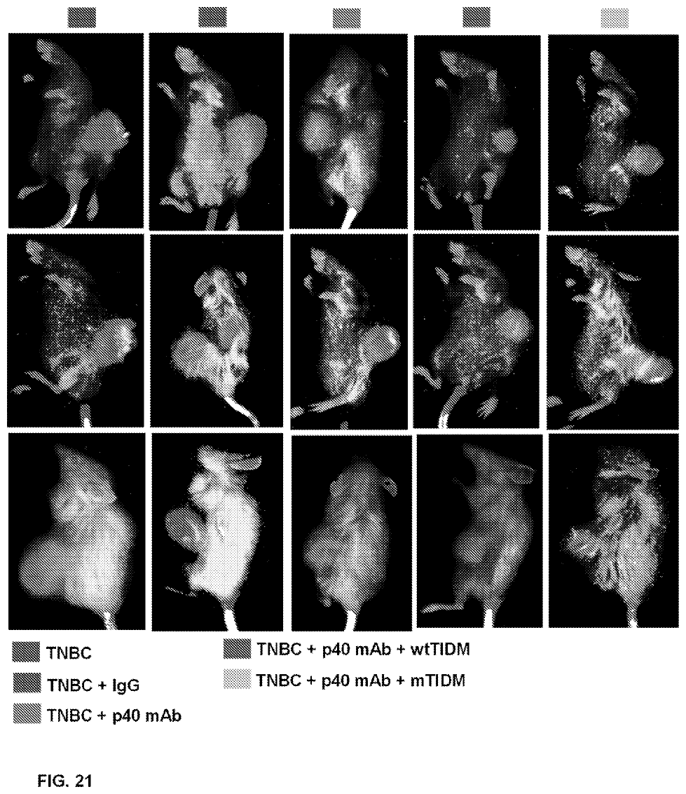

Daily intranasal administration of wild type TLR2-interacting domain of MyD88 (wtTIDM) peptide significantly stimulated the p40 mAb a3-3a-mediated reduction of TNBC tumor in a patient-derived xenograft (PDX) model in mice (FIG. 21). This result was specific as mutated (m) TIDM peptide remained unable to stimulate the p40 mAb-mediated reduction of TNBC tumor (FIG. 21).

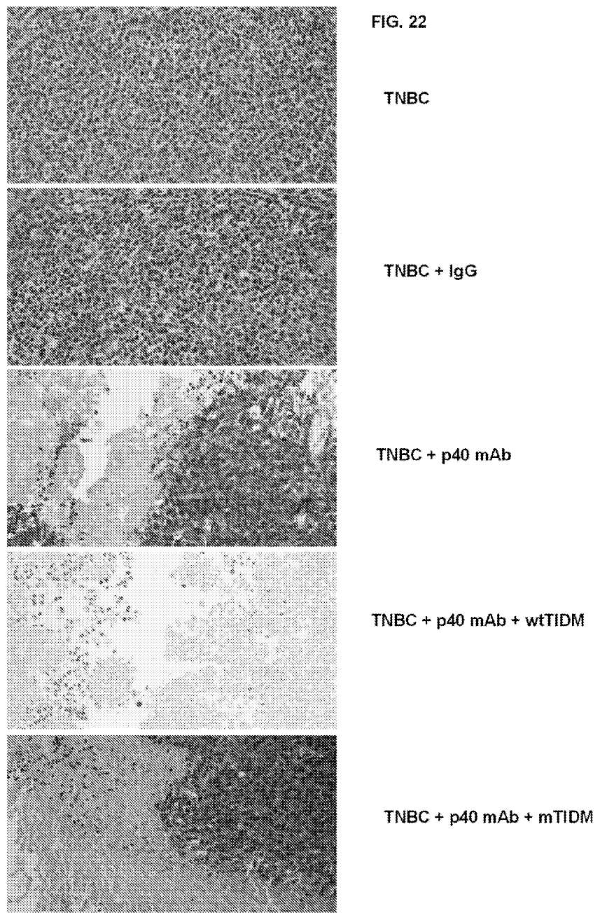

As evident from FIG. 22, intranasal administration of wtTIDM, but not mTIDM, peptide stimulated the death of TNBC tumor in p40 mAb a3-3a-treated patient-derived xenograft (PDX) model in mice. Very few live cells were seen in the core of TNBC tumor of PDX mice that were treated with the combination wtTIDM peptide and p40 mAb a3-3a (FIG. 22).

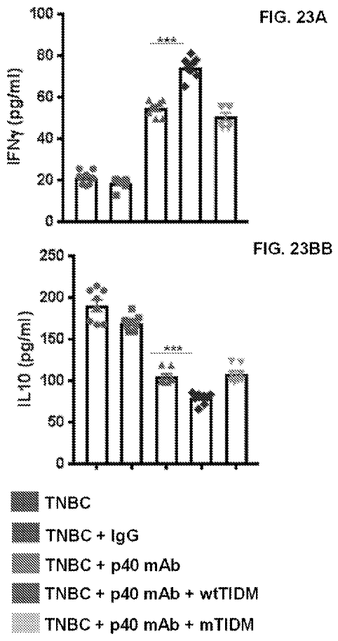

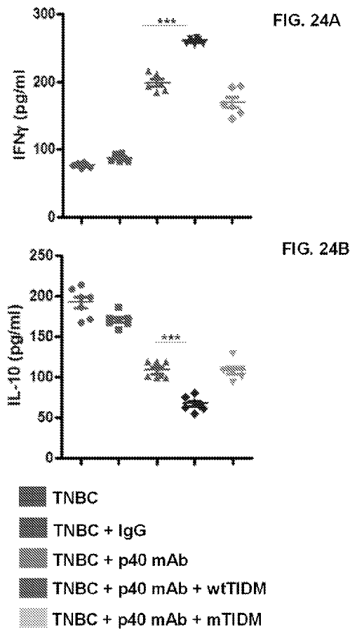

Intranasal administration of wtTIDM, but not mTIDM, peptide stimulated p40 mAb a3-3a-mediated induction of IFNγ in tumor tissues (FIG. 23A) and serum (FIG. 24A) of PDX mouse model of TNBC.

Intranasal administration of wtTIDM, but not mTIDM, peptide potentiated p40 mAb a3-3a-mediated reduction of IL-10 in tumor tissues (FIG. 23B) and serum (FIG. 24B) of PDX mouse model of TNBC.

In summary, we have demonstrated the following:

A. Human TNBC cells produce more p40 than p40₂.

B. Neutralizing monoclonal antibodies against p40 (a3-3a) induced death in human triple-negative breast cancer (BT-549 and HCC70) cells.

C. Neutralizing monoclonal antibodies against p40 (a3-7g) induced death in human prostate (LnCAP), hepatic (Hep3B) and triple-negative breast (HCC70) cancer cells.

D. Weekly treatment of p40 mAb a3-3a led to regression of tumor in a PDX mouse model of TNBC.

E. The p40 mAb A3-3A treatment increased cancer-destroying Th1 and Tc1 immune responses in TNBC mice.

F. In the presence of wtNBD peptide, the p40 mAb a3-3a caused significant regression of tumor in a PDX mouse model of TNBC.

G. The combination of wtNBD peptide and p40 mAb a3-3a led to remarkable reduction of tumor in a PDX mouse model of TNBC.

H. Intranasal administration of either wtTIDM or wtNBD peptide led to greater Tc1 response in p40 mAb-treated TNBC mice.

Discussion

TNBC is an aggressive type of breast cancer associated with limited treatment options and as a result, TNBC accounts for 5% of all cancer-related deaths annually. With current therapies, the median overall survival for TNBC is 10.2 months with a 5-year survival rate of ˜65% for local tumors and 11% for those with tumor spreading to distant organs. Since TNBC is chemotherapy sensitive, chemotherapy is the standard of care, especially in cases where surgery is not an option. Recently, a number of immunotherapies in combination with different investigational drugs are also being tested for TNBC [31, 32]. IL-12 is an important cytokine for eliciting cell-mediated immune response [6]. Antigen-presenting cells upon activation through Toll-like receptors and/or interactions with CD4⁺ T cells produce this heterodimeric (p35:p40) cytokine [6, 33]. Although IL-12 p40 monomer (p40) was known as biologically inactive, recently we have seen greater levels of p40 in serum of prostate cancer patients as compared to healthy controls and regression of prostate tumor in mice after neutralization of p40 by a specific mAb [4]. Interestingly, we have seen opposite results in serum of multiple sclerosis (MS) patients in which the level of p40 in lower in serum of MS patients as compared to healthy controls and supplementation of p40 inhibits autoimmune demyelination in mice [5]. Here, we demonstrate that the level of p40 is significantly higher in serum of breast cancer patients as compared to age-matched healthy controls and that human TNBC cells as well as PDX mouse model of TNBC also produce excess p40. Accordingly, mAb-mediated neutralization of p40 induces death in human TNBC cells and suppresses tumor growth in a PDX mouse model of TNBC. These results indicate the possible immunotherapeutic prospect of p40 mAb in TNBC.

To achieve tumor regression, it is almost mandatory to induce apoptosis and/or necrosis in tumor tissues. From several angles, we have demonstrated greater death response in TNBC tumor after p40 mAb treatment. Our conclusion is dependent on the following observations: First, H&E staining showed an empty or a dead tumor core in p40 mAb-treated PDX mice. On the other hand, we did not notice such hollow core in tumors of either untreated or control IgG-treated PDX mice. Second, as expected, we found marked upregulation of apoptosis-related genes such as cytochrome C, caspase 3, caspase 8, caspase 9, p53, BAD, BID, BAX, and BAK in tumor tissues of p40 mAb-treated PDX mice as compared to that of either untreated or control IgG-treated PDX mice. Third, number of TUNEL-positive cells was much higher in tumors of p40 mAb-treated PDX mice than that of either untreated or control IgG-treated PDX mice. Fourth, dual FACS staining with PI and annexin V revealed increase in early apoptotic (PI negative and annexin V positive), late apoptotic (PI positive and annexin V positive) as well as necrotic (PI positive and annexin V negative) cells in tumors of p40 mAb-treated PDX mice as compared to untreated or control IgG-treated PDX mice. Therefore, p40 mAb may be considered for inducing cell death response in TNBC tumors.

Upregulation of CD8+ cytotoxic-1 T (Tc1) lymphocyte-mediated adaptive immune response is one of the important mechanisms for the induction of death response in different cancers including TNBC. Accordingly, Tc1 cells capable of producing IFNγ are a major immunological effector cell population mediating resistance to cancer [34, 35]. Tc1 cells can also eradicate the growth and metastasis of malignant tumor cells [35]. However, in clinical trials, only a limited number of patients respond to Tc1 cell therapy [36]. Although underlying mechanisms are unknown, it is probably due to lacking of T cell helper arm [21, 22]. Similar to Tc1 cells, CD4+T helper 1 (Th1) cells that are essential for generating cellular immunity do not directly kill tumor cells. However, Th1 cells play an important role in priming Tc1-mediated antitumor responses [21]. It is possible that Th1 cells provide IL-2 needed to elicit Tc1-mediated anti-tumor immunity [20]. It has been also reported that Th1 cells play an important role in the induction of Tc1 cell responses through the activation of DC via CD40 ligation [37]. Moreover, Th1 cells are required in defining the magnitude and persistence of Tc1 responses and for Tc1 infiltration into tumors [38, 39]. Therefore, upregulation of both Tc1 and Th1 responses together is important for successful cancer immunotherapy. It is nice to see that p40 mAb treatment markedly upregulates both Th1 and Tc1 responses in spleen and TNBC tumors of PDX mice.

It is known that blood monocytes infiltrate into tumors to be ultimately differentiated into macrophages, known as tumor-associated macrophages (TAMs) [40, 41]. TAMs can be divided into specific subsets based on marker, function and phenotype. For example, it is widely accepted that arginase 1-expressing and polyamine-producing tumor-associated M2 macrophages (TAM2) support pro-oncogenic functions and are pathogenic in cancer [41, 42]. On the other hand, being characterized by the expression of inducible nitric oxide synthase (iNOS) and tumor necrosis factor α (TNFα), tumor-associated M1 macrophages (TAM1) are known to exhibit anti-cancer activity via proinflammatory immune responses [42]. Therefore, reprogramming immunosuppressive TAM2 towards an pro-inflammatory TAM1 state is known to limit tumor growth [43]. Accordingly, we have seen very few TAM1 and many TAM2 in TNBC tumors of untreated PDX mice. However, p40 mAb immunotherapy markedly upregulated TAM1 and downregulated TAM2 in TNBC tumors of PDX mice. TAMs are also known to modulate PD-1/PD-L1 signaling in which TAM-secreted cytokines induce the expression of PD-1 and PD-L1 [44, 45]. Numerous studies highlight an important role of PD-1/PD-L1 in tumor progression via inhibition of immune responses, stimulation of apoptosis of antigen-specific T cells, and suppression of apoptosis of regulatory T cells [46-48]. According to Sun et al [49], PD-1(+) immune cell infiltration is inversely correlated with survival of operable breast cancer patients. According to Zhu et al [50], although CSF1/CSF1R blockade reprograms tumor-infiltrating macrophages and improves response to T-cell checkpoint immunotherapy in pancreatic cancer models, tumor regression is restricted by simultaneous upregulation of PD-L1. Together, for an immunotherapy to be successful in cancer treatment, it is important to overcome the resistance from PD-1/PD-L1 signaling. It is nice to see that consistent to the promotion of TAM1 and suppression of TAM2, p40 mAb treatment markedly suppressed the PD-1/PD-L1 axis in TNBC tumors. Therefore, p40 mAb immunotherapy is capable of suppressing different death-evading signaling pathways in TNBC tumors.

### CONCLUSION

In summary, here, we delineate upregulation of p40 in breast cancer patients, human TNBC cells and PDX mouse model of TNBC and that scavenging of p40 by p40 mAb enriches anti-oncogenic Tc1 and Th1 cells, mitigates pro-oncogenic Th2 cells, upregulates M1 macrophages, suppresses PD-1/PD-L1 signaling, and induces apoptosis and/or necrosis, leading to tumor regression in a PDX mouse model of TNBC. Although the diverse disease process of human TNBC is not precisely the same as PDX mouse model of TNBC, our results suggest that p40 mAb may provide a new immunotherapeutic avenue against TNBC.

Methods

Reagents: Human TNBC cell lines (BT-549 and HCC70) were purchased from ATCC. Cell culture materials (RPMI 1640, DMEM, L-glutamine, antibiotic/antimycotic) were purchased from Life Technologies. Hamster IgG (cat # IR-HT-GF) was obtained from Innovative Research. MTT assay kit (cat # CGD1), and LDH assay kit (cat # TOX7) were purchased from Sigma. TUNEL assay kit (cat #QIA39) was purchased from Calbiochem and Annexin V assay kit (cat # K101-25) was purchased from Biovision. ELISA kits for human IFN-γ, IL-12, IL-23, and IL-10 were purchased from ThermoFisher. Antibody against inducible nitric oxide synthase (iNOS) were purchased from BD Bioscience. Antibodies against ionized calcium binding adaptor molecule 1 (Iba1), PD-1, and PD-L1 were purchased from Abcam. Antibody against arginase 1 was purchased from Thermo Fisher.

Serum samples of breast cancer patients: Serum samples of pretreated breast cancer patients and age-matched healthy controls were obtained from Discovery Life Sciences, Los Osos, Calif.

Animals: Animal maintaining and experiments were in accordance with National Institute of Health guidelines and were approved by the Institutional Animal Care and Use committee of the Rush University of Medical Center, Chicago, Ill. Patient-derived xenograft (PDX) model (ID # TM00096) was purchased from Jackson Laboratory, Bar Harbor, Me., USA. In this model, TNBC tumor fragments at passage P1-P9 (invasive ductal carcinoma; TNBC ER⁻PR⁻HER2⁻) were engrafted in the flank of female 6-8 week old NOD scid gamma (NSG) mice. Mice were shipped to us by Jackson Laboratory within ˜2 weeks following tumor engraftment. PDX mice were maintained in our temperature-controlled animal vivarium with adequate food and water.

Tumor measurement: Tumor growth was measured with a caliper and tumor cross-sectional area was determined with the formula (mm²=longest diameter X shortest diameter). Treatment with p40 mAb started when the tumor sizes reached 0.6-0.8 cm² in area. The p40 mAb a3-3a was injected once a week intraperitoneally in 0.1 ml volume of sterile PBS-1% normal mouse serum. The tumors were then measured to determine progression or regression. Infrared dye (Alexa 800-conjugated 2DG dye; Licor) was injected via tail-vein on the day before imaging analysis. Mice were sacrificed at the end of the study and tumor tissues were collected for different biochemical analyses.

Sandwich ELISA: Sandwich ELISA was used to quantify p40₂ and p40 as described by us [10, 11]. Briefly, for p40₂, mAb a3-1d (1.3 mg/mL) was diluted 1:3000 and added to each well (100 μL/well) of a 96-well ELISA plate for coating. The biotinylated p40₂ mAb d7-12c (2 mg/mL) was diluted 1:3000 and used as detection antibody. Similarly for p40, mAb a3-3a (1.3 mg/mL) and biotinylated p40 mAb a3-7g (2 mg/mL) were also diluted 1:3000 and used as coating and detection antibodies, respectively [10]. Concentrations of IFN-γ, IL-12, IL-23, and IL-10 were measured in serum or tissue homogenates by ELISA (eBioscience/ThermoFisher), according to the manufacturer's instructions as described before [5].

Isolation of splenocytes: Spleens isolated from treated or untreated PDX mice were placed into a cell strainer and mashed with a syringe plunger. Resulting single-cell suspensions were treated with RBC lysis buffer (Sigma-Aldrich), washed, and cultured in RPMI 1640 supplemented with 10% FBS, 50 μM 2-ME, 2 mM L-glutamine, 100 U/ml penicillin, and 100 μg/ml streptomycin.

Flow cytometry: Single-cell suspensions isolated from mouse spleen or tumor were stained with Zombie Aqua™ Fixable Viability Kit (Biolegend) according to the manufacturer's instructions. Cells were washed with FACS buffer (ThermoFisher) and stained with FITC-anti-human CD4 antibody and APC/Cy7-anti-human CD8 antibody (Biolegend) for extracellular stains. For IFNγ staining, cells were stained with PE-anti-human IFNγ antibody (Biolegend) and detected by flow cytometry analysis. Only PE-treated and unstained cells served as control. Flow cytometric analyses were performed using the LSRFortessa analyzer (BD Biosciences) and analyzed using the FlowJo Software (v10) as described [4, 5].

Tissue preparation and Immunohistochemistry: Paraffin embedded tissue sections were prepared and tissue sections were cut 5 micron in size as described [11, 12]. To eliminate endogenous peroxidase activity, tissue sections were deparaffinized, rehydrated and incubated with 3% H₂O₂ in methanol for 15 min at room temperature. Antigen retrieval was performed at 95° C. for 20 min by placing the slides in 0.01 M sodium citrate buffer (pH 6.0). After blocking, the slides were then incubated with the primary antibodies against CD8 and IFNγ for 2 h at room temperature followed by washing and incubation with Cy2 or Cy5 (Jackson ImmunoResearch Laboratories, West Grove, Pa.) secondary antibodies at room temperature for 1 h [13].

Cell Viability Measurements:

MTT assay: Mitochondrial activity was measured with the 3-(4, 5-dimethylthiazol-2-yl)-2, 5-diphenyltetrazolium bromide (MTT) assay (Sigma) as described before [14, 15]. The cells were grown on 24-well culture plates with 500 μl of medium and treated with various reagents according to the experimental design. At the end of the treatment period, 300 μl of culture medium were removed from each well, and 20 μl of MTT solution (5 mg/ml) were added and incubated for 1 h.

LDH assay: The activity of lactate dehydrogenase (LDH) was measured using the direct spectrophotometric assay using an assay kit from Sigma as described before [14, 15].

Liver toxicity assay: The activity of alanine aminotransferase (ALT) or serum glutamic-pyruvic transaminase (SGPT) was monitored in serum using an assay kit from Sigma following manufacturer's protocol.

TUNEL and Actin double-labeling: Following treatments with p40 mAb, TUNEL assays were performed as described earlier [16, 17]. Briefly, tumor tissue sections were blocked using blocking buffer followed by treatment with 20 μg/ml proteinase K at room temperature and one wash with PBS. Next, the samples were incubated for 90 min in terminal deoxynucleotidyl transferase (TdT) equilibration buffer containing anti-Actin antibody. After three washes in PBST, the sections were incubated in fluorescein-fragEL TdT reaction mix containing TdT enzyme and secondary antibody for 60 min at 37° C. Prior to mounting, the samples were washed twice in PBS. Finally, the samples were mounted using mounting media containing 4,6,-DiAmidino-2-PhenylIndole (DAPI), which allows the visualization of total cell population and observed for fluorescein-labeled DNA fragments.

Real-time PCR: Total RNA was isolated from tumor tissues using Ultraspec II RNA Reagent (Biotecx Laboratories Inc.) according to the manufacturer's protocol. To remove any contaminating genomic DNA, total RNA was digested with DNase. Then DNase-digested RNA was analyzed by real-time PCR in the ABI-Prism7700 Sequence Detection System (Applied Biosystems) as described previously [4, 5].

Statistical Analysis: For tumor regression, quantitative data were presented as the mean±SEM. Statistical significance were accessed via one-way ANOVA with Student-Newman-Keuls posthoc analysis. Other data were expressed as means±SD of three independent experiments. Statistical differences between means were calculated by the Student's t-test. A p-value of less than 0.05 (p<0.05) was considered statistically significant.

TABLE 1
List of primers used in this study

| Gene | Directions | Sequence (5’...3’) | SEQ ID NO: |
|---|---|---|---|
| GAPDH | Sense | GCATCTTCTTGTGCAGTGCC | 10 |
| | Antisense | TACGGCCAAATCCGTTCACA | 11 |
| Cytochrome C | Sense | CCCCCAGCCTCCCTTATCTT | 12 |
| | Antisense | GGTCTGCCCTTTCTCCCTTC | 13 |
| Caspase 3 | Sense | GAGCTTGGAACGGTACGCTA | 14 |
| | Antisense | CCGTACCAGAGCGAGATGAC | 15 |
| Caspase 8 | Sense | AACATTCGGAGGCATTTCTGT | 16 |
| | Antisense | AGAAGAGCTGTAACCTGTGGC | 17 |
| Caspase 9 | Sense | CTCTGAAGACCTGCAGTCCC | 18 |
| | Antisense | CTGCTCCACATTGCCCTACA | 19 |
| P53 | Sense | ACCAGGGCAACTATGGCTTC | 20 |
| | Antisense | AGTGGATCCTGGGGATTGTG | 21 |
| BAD | Sense | CAGCGTACGCACACCTATCC | 22 |
| | Antisense | CGGGATCGGACTTCCTCAAG | 23 |
| BID | Sense | TCTGAGGTCAGCAACGGTTC | 24 |
| | Antisense | TTTGTCTTCCTCCGACAGGC | 25 |
| BAX | Sense | CTGGATCCAAGACCAGGGTG | 26 |
| | Antisense | CCTTTCCCCTTCCCCCATTC | 27 |
| BCL2 | Sense | AGCATGCGACCTCTGTTTGA | 28 |
| | Antisense | GCCACACGTTTCTTGGCAAT | 29 |
| BCL-XL | Sense | TTGTACCTGCTTGCTGGTCG | 30 |
| | Antisense | CCCGGTTGCTCTGAGACATT | 31 |
| BAK | Sense | CCTGGGCCAACACGC | 32 |
| | Antisense | CTGTGGGCTGAAGCTGTTCTA | 33 |

While only certain embodiments have been set forth, alternatives and modifications will be apparent from the above description to those skilled in the art. These and other alternatives are considered equivalents and within the spirit and scope of this disclosure and the appended claims.

### REFERENCES

1. Siegel R L, Miller K D, Jemal A: Cancer Statistics, 2017. CA Cancer J Clin 2017, 67(1):7-30.
2. Bianchini G, Balko J M, Mayer I A, Sanders M E, Gianni L: Triple-negative breast cancer: challenges and opportunities of a heterogeneous disease. Nat Rev Clin Oncol 2016, 13(11):674-690.
3. Carey L, Winer E, Viale G, Cameron D, Gianni L: Triple-negative breast cancer: disease entity or title of convenience? Nat Rev Clin Oncol 2010, 7(12):683-692.
4. Kundu M, Roy A, Pahan K: Selective neutralization of IL-12 p40 monomer induces death in prostate cancer cells via IL-12-IFN-gamma. Proc Natl Acad Sci USA 2017, 114(43):11482-11487.
5. Mondal S, Kundu M, Jana M, Roy A, Rangasamy S B, Modi K K, Wallace J, Albalawi Y A, Balabanov R, Pahan K: IL-12 p40 monomer is different from other IL-12 family members to selectively inhibit IL-12Rbeta1 internalization and suppress EAE. Proc Natl Acad Sci USA 2020, 117(35):21557-21567.
6. Gately M K, Renzetti L M, Magram J, Stern A S, Adorini L, Gubler U, Presky D H: The interleukin-12/interleukin-12-receptor system: role in normal and pathologic immune responses. Annu Rev Immunol 1998, 16:495-521.
7. Cua D J, Sherlock J, Chen Y, Murphy C A, Joyce B, Seymour B, Lucian L, To W, Kwan S, Churakova T et al: Interleukin-23 rather than interleukin-12 is the critical cytokine for autoimmune inflammation of the brain. Nature 2003, 421(6924):744-748.
8. Brahmachari S, Pahan K: Role of cytokine p40 family in multiple sclerosis. Minerva Med 2008, 99(2):105-118.
9. Jana M, Dasgupta S, Pal U, Pahan K: IL-12 p40 homodimer, the so-called biologically inactive molecule, induces nitric oxide synthase in microglia via IL-12R beta 1. Glia 2009, 57(14):1553-1565.
10. Dasgupta S, Bandopadhyay M, Pahan K: Generation of functional blocking monoclonal antibodies against mouse interleukin-12 p40 homodimer and monomer. Hybridoma (Larchmt) 2008, 27(3):141-151.
11. Mondal S, Roy A, Pahan K: Functional blocking monoclonal antibodies against IL-12p40 homodimer inhibit adoptive transfer of experimental allergic encephalomyelitis. J Immunol 2009, 182(8):5013-5023.
12. Mondal S, Pahan K: Cinnamon ameliorates experimental allergic encephalomyelitis in mice via regulatory T cells: implications for multiple sclerosis therapy. PLoS One 2015, 10(1):e0116566.
13. Ghosh A, Roy A, Liu X, Kordower J H, Mufson E J, Hartley D M, Ghosh S, Mosley R L, Gendelman H E, Pahan K: Selective inhibition of N F-kappaB activation prevents dopaminergic neuronal loss in a mouse model of Parkinson's disease. Proc Natl Acad Sci USA 2007, 104(47):18754-18759.
14. Corbett G T, Roy A, Pahan K: Gemfibrozil, a lipid-lowering drug, upregulates IL-1 receptor antagonist in mouse cortical neurons: implications for neuronal self-defense. J Immunol 2012, 189(2):1002-1013.
15. Jana A, Pahan K: Fibrillar amyloid-beta-activated human astroglia kill primary human neurons via neutral sphingomyelinase: implications for Alzheimer's disease. J Neurosci 2010, 30(38):12676-12689.
16. Modi K K, Roy A, Brahmachari S, Rangasamy S B, Pahan K: Cinnamon and Its Metabolite Sodium Benzoate Attenuate the Activation of p21rac and Protect Memory and Learning in an Animal Model of Alzheimer's Disease. PLoS One 2015, 10(6):e0130398.
17. Rangasamy S B, Jana M, Roy A, Corbett G T, Kundu M, Chandra S, Mondal S, Dasarathi S, Mufson E J, Mishra R K et al: Selective disruption of TLR2-MyD88 interaction inhibits inflammation and attenuates Alzheimer's pathology. J Clin Invest 2018, 128(10):4297-4312.
18. Leonard J P, Waldburger K E, Goldman S J: Prevention of experimental autoimmune encephalomyelitis by antibodies against interleukin 12. J Exp Med 1995, 181(1):381-386.
19. Chen Y, Langrish C L, McKenzie B, Joyce-Shaikh B, Stumhofer J S, McClanahan T, Blumenschein W, Churakovsa T, Low J, Presta L et al: Anti-IL-23 therapy inhibits multiple inflammatory pathways and ameliorates autoimmune encephalomyelitis. J Clin Invest 2006, 116(5):1317-1326.
20. Fearon E R, Pardoll D M, Itaya T, Golumbek P, Levitsky H I, Simons J W, Karasuyama H, Vogelstein B, Frost P: Interleukin-2 production by tumor cells bypasses T helper function in the generation of an antitumor response. Cell 1990, 60(3):397-403.
21. Goedegebuure P S, Eberlein T J: The role of CD4+ tumor-infiltrating lymphocytes in human solid tumors. Immunol Res 1995, 14(2):119-131.
22. Pockaj B A, Sherry R M, Wei J P, Yannelli J R, Carter C S, Leitman S F, Carasquillo J A, Steinberg S M, Rosenberg S A, Yang J C: Localization of 111indium-labeled tumor infiltrating lymphocytes to tumor in patients receiving adoptive immunotherapy. Augmentation with cyclophosphamide and correlation with response. Cancer 1994, 73(6):1731-1737.
23. Li J, Jie H B, Lei Y, Gildener-Leapman N, Trivedi S, Green T, Kane L P, Ferris R L: PD-1/SHP-2 inhibits Tc1/Th1 phenotypic responses and the activation of T cells in the tumor microenvironment. Cancer Res 2015, 75(3):508-518.
24. Coleman C B, Lang J, Sweet L A, Smith N A, Freed B M, Pan Z, Haverkos B, Pelanda R, Rochford R: Epstein-Barr Virus Type 2 Infects T Cells and Induces B Cell Lymphomagenesis in Humanized Mice. J Virol 2018, 92(21).
25. Serr I, Furst R W, Achenbach P, Scherm M G, Gokmen F, Haupt F, Sedlmeier E M, Knopff A, Shultz L, Willis R A et al: Type 1 diabetes vaccine candidates promote human Foxp3(+)Treg induction in humanized mice. Nat Commun 2016, 7:10991.
26. Rosato R R, Davila-Gonzalez D, Choi D S, Qian W, Chen W, Kozielski A J, Wong H, Dave B, Chang J C: Evaluation of anti-PD-1-based therapy against triple-negative breast cancer patient-derived xenograft tumors engrafted in humanized mouse models. Breast Cancer Res 2018, 20(1):108.
27. Najima Y, Tomizawa-Murasawa M, Saito Y, Watanabe T, Ono R, Ochi T, Suzuki N, Fujiwara H, Ohara O, Shultz L D et al: Induction of WT1-specific human CD8+ T cells from human HSCs in HLA class I Tg NOD/SCID/IL2rgKO mice. Blood 2016, 127(6):722-734.
28. Yuan Z Y, Luo R Z, Peng R J, Wang S S, Xue C: High infiltration of tumor-associated macrophages in triple-negative breast cancer is associated with a higher risk of distant metastasis. Onco Targets Ther 2014, 7:1475-1480.
29. Santoni M, Romagnoli E, Saladino T, Foghini L, Guarino S, Capponi M, Giannini M, Cognigni P D, Ferrara G, Battelli N: Triple negative breast cancer: Key role of Tumor-Associated Macrophages in regulating the activity of anti-PD-1/PD-L1 agents. Biochim Biophys Acta Rev Cancer 2018, 1869(1):78-84.
30. Han Y, Liu D, Li L: PD-1/PD-L1 pathway: current researches in cancer. Am J Cancer Res 2020, 10(3):727-742.
31. Emens L A: Breast Cancer Immunotherapy: Facts and Hopes. Clin Cancer Res 2018, 24(3):511-520.
32. Marra A, Viale G, Curigliano G: Recent advances in triple negative breast cancer: the immunotherapy era. BMC Med 2019, 17(1):90.
33. Ngiow S F, Teng M W, Smyth M J: A balance of interleukin-12 and -23 in cancer. Trends Immunol 2013, 34(11):548-555.
34. Mukai S, Kjaergaard J, Shu S, Plautz G E: Infiltration of tumors by systemically transferred tumor-reactive T lymphocytes is required for antitumor efficacy. Cancer Res 1999, 59(20):5245-5249.
35. Tanaka H, Yoshizawa H, Yamaguchi Y, Ito K, Kagamu H, Suzuki E, Gejyo F, Hamada H, Arakawa M: Successful adoptive immunotherapy of murine poorly immunogenic tumor with specific effector cells generated from gene-modified tumor-primed lymph node cells. J Immunol 1999, 162(6):3574-3582.
36. Chang A E, Yoshizawa H, Sakai K, Cameron M J, Sondak V K, Shu S: Clinical observations on adoptive immunotherapy with vaccine-primed T-lymphocytes secondarily sensitized to tumor in vitro. Cancer Res 1993, 53(5):1043-1050.
37. Bennett S R, Carbone F R, Karamalis F, Flavell R A, Miller J F, Heath W R: Help for cytotoxic-T-cell responses is mediated by CD40 signalling. Nature 1998, 393(6684):478-480.
38. Marzo A L, Kinnear B F, Lake R A, Frelinger J J, Collins E J, Robinson B W, Scott B: Tumor-specific CD4+ T cells have a major “post-licensing” role in CTL mediated anti-tumor immunity. J Immunol 2000, 165(11):6047-6055.
39. Marzo A L, Lake R A, Robinson B W, Scott B: T-cell receptor transgenic analysis of tumor-specific CD8 and CD4 responses in the eradication of solid tumors. Cancer Res 1999, 59(5):1071-1079.
40. Yona S, Kim K W, Wolf Y, Mildner A, Varol D, Breker M, Strauss-Ayali D, Viukov S, Guilliams M, Misharin A et al: Fate mapping reveals origins and dynamics of monocytes and tissue macrophages under homeostasis. Immunity 2013, 38(1):79-91.
41. Van Overmeire E, Laoui D, Keirsse J, Bonelli S, Lahmar Q, Van Ginderachter J A: STAT of the union: dynamics of distinct tumor-associated macrophage subsets governed by STAT1. Eur J Immunol 2014, 44(8):2238-2242.
42. Qian B Z, Pollard J W: Macrophage diversity enhances tumor progression and metastasis. Cell 2010, 141(1):39-51.
43. Pyonteck S M, Akkari L, Schuhmacher A J, Bowman R L, Sevenich L, Quail D F, Olson O C, Quick M L, Huse J T, Teijeiro V et al: CSF-1R inhibition alters macrophage polarization and blocks glioma progression. Nat Med 2013, 19(10):1264-1272.
44. Santarpia M, Gonzalez-Cao M, Viteri S, Karachaliou N, Altavilla G, Rosell R: Programmed cell death protein-1/programmed cell death ligand-1 pathway inhibition and predictive biomarkers: understanding transforming growth factor-beta role. Transl Lung Cancer Res 2015, 4(6):728-742.
45. Zhang X, Zeng Y, Qu Q, Zhu J, Liu Z, Ning W, Zeng H, Zhang N, Du W, Chen C et al: PD-L1 induced by IFN-gamma from tumor-associated macrophages via the JAK/STAT3 and PI3K/AKT signaling pathways promoted progression of lung cancer. Int J Clin Oncol 2017, 22(6):1026-1033.
46. Arlauckas S P, Garris C S, Kohler R H, Kitaoka M, Cuccarese M F, Yang K S, Miller M A, Carlson J C, Freeman G J, Anthony R M et al: In vivo imaging reveals a tumor-associated macrophage-mediated resistance pathway in anti-PD-1 therapy. Sci Transl Med 2017, 9(389).
47. Ljunggren H G, Jonsson R, Hoglund P: Seminal immunologic discoveries with direct clinical implications: The 2018 Nobel Prize in Physiology or Medicine honours discoveries in cancer immunotherapy. Scand J Immunol 2018, 88(6):e12731.
48. Messenheimer D J, Jensen S M, Afentoulis M E, Wegmann K W, Feng Z, Friedman D J, Gough M J, Urba W J, Fox B A: Timing of PD-1 Blockade Is Critical to Effective Combination Immunotherapy with Anti-OX40. Clin Cancer Res 2017, 23(20):6165-6177.
49. Sun S, Fei X, Mao Y, Wang X, Garfield D H, Huang O, Wang J, Yuan F, Sun L, Yu Q et al: PD-1(+) immune cell infiltration inversely correlates with survival of operable breast cancer patients. Cancer Immunol Immunother 2014, 63(4):395-406.
50. Zhu Y, Knolhoff B L, Meyer M A, Nywening T M, West B L, Luo J, Wang-Gillam A, Goedegebuure S P, Linehan D C, DeNardo D G: CSF1/CSF1R blockade reprograms tumor-infiltrating macrophages and improves response to T-cell checkpoint immunotherapy in pancreatic cancer models. Cancer Res 2014, 74(18):5057-5069.

1. A method for treating triple negative breast cancer (TNBC), the method comprising administering to a subject in need of such treatment a composition comprising a therapeutically effective amount of an antibody against p40 monomer or an immunologically active fragment thereof.

2. (canceled)

3. The method according to claim 1, wherein the antibody against p40 monomer or an immunologically active fragment thereof is selected from the group consisting of polyclonal, monoclonal, human, humanized, and chimeric antibodies; single chain antibodies; and epitope-binding antibody fragments.

4. (canceled)

5. The method according to claim 1, wherein the composition further comprises a peptide comprising a TLR2-interacting domain of MyD88 (TIDM).

6. The method according to claim 5, wherein the TIDM peptide comprises the sequence PGAHQK (SEQ ID NO: 1).

7. (canceled)

8. The method according to claim 5, wherein the TIDM peptide further comprises an Antennapedia homeodomain, wherein the Antennapedia homeodomain links to an amino terminus or a carboxy terminus of the TIDM peptide.

9. (canceled)

10. The method according to claim 5, wherein the TIDM peptide sequence is drqikiwfqnrrmkwkkPGAHQK (SEQ ID NO: 3).

11. The method according to claim 1, wherein the composition further comprises a peptide comprising a NEMO-binding domain (NBD).

12. The method according to claim 11, wherein the NBD peptide comprises the sequence LDWSWL (SEQ ID NO: 6).

13. (canceled)

14. The method according to claim 11, wherein the NBD peptide further comprises an Antennapedia homeodomain, wherein the Antennapedia homeodomain links to an amino terminus or a carboxy terminus of the NBD peptide.

15. (canceled)

16. The method according to claim 11, wherein the NBD peptide sequence is drqikiwfqnrrmkwkkLDWSWL (SEQ ID NO: 7).

17. A method for treating a cancer, the method comprising administering to a subject in need of such treatment a composition comprising a therapeutically effective amount of an antibody against p40 monomer or an immunologically active fragment thereof and a binding domain peptide.

18. The method according to claim 17, wherein the binding domain peptide comprises a TLR2-interacting domain of MyD88 (TIDM) peptide or a NEMO-binding domain (NBD) peptide.

19. The method according to claim 18, wherein the TIDM peptide comprises the sequence PGAHQK (SEQ ID NO: 1).

20. The method according to claim 18, wherein the NBD peptide comprises the sequence LDWSWL (SEQ ID NO: 6).

21. (canceled)

22. The method according to claim 17, wherein the TIDM peptide or the NBD peptide further comprises an Antennapedia homeodomain.

23. The method according to claim 22, wherein the Antennapedia homeodomain links to an amino terminus or a carboxy terminus of the TIDM peptide or the NBD peptide.

24. The method according to claim 22, wherein the TIDM peptide sequence is drqikiwfqnrrmkwkkPGAHQK (SEQ ID NO: 3).

25. The method according to claim 15, wherein the NBD peptide sequence is drqikiwtqnrrmkwkkLDWSWL (SEQ ID NO: 7).

26. (canceled)

27. The method according to claim 17, wherein the antibody against p40 monomer or an immunologically active fragment thereof is selected from the group consisting of polyclonal, monoclonal, human, humanized, and chimeric antibodies; single chain antibodies; and epitope-binding antibody fragments.

28. (canceled)

29. The method according to claim 17, wherein the cancer is selected from the group consisting of prostate cancer, non-TNBC types of breast cancer, pancreatic cancer, liver cancer, ovarian cancer, and other cancers that overexpress p40 monomer.


SEQUENCE LISTING (33 sequences)

SEQ ID NO: 1 - protein, 6 aa, Artificial Sequence
  TIDM peptide
PGAHQK

SEQ ID NO: 2 - protein, 17 aa, Artificial Sequence
  Antennapedia homeodomain peptide
DRQIKIWFQNRRMKWKK

SEQ ID NO: 3 - protein, 23 aa, Artificial Sequence
  TIDM peptide plus Antennapedia homeodomain peptide
DRQIKIWFQNRRMKWKKPGAHQK

SEQ ID NO: 4 - protein, 6 aa, Artificial Sequence
  Mutated TIDM peptide
PGWHGD

SEQ ID NO: 5 - protein, 23 aa, Artificial Sequence
  Mutated TIDM peptide plus Antennapedia homeodomain
DRQIKIWFQNRMIKWKKPGWHGD

SEQ ID NO: 6 - protein, 6 aa, Artificial Sequence
  NBD peptide
LDWSWL

SEQ ID NO: 7 - protein, 23 aa, Artificial Sequence
  NBD peptide plus Antennapedia homeodomain peptide
DRQIKIWFQNRRMKWKKLDWSWL

SEQ ID NO: 8 - protein, 6 aa, Artificial Sequence
  mutated NBD peptide
LDASAL

SEQ ID NO: 9 - protein, 23 aa, Artificial Sequence
  mutated NBD peptide plus Antennapedia homeodomain peptide
DRQIKIWFQNRRMKWKKLDASAL

SEQ ID NO: 10 - DNA, 20 bp, Artificial Sequence
  GADPH sense primer
gcatcttcttgtgcagtgcc

SEQ ID NO: 11 - DNA, 20 bp, Artificial Sequence
  GADPH antisense primer
tacggccaaatccgttcaca

SEQ ID NO: 12 - DNA, 20 bp, Artificial Sequence
  Cytochrome C sense primer
cccccagcctcccttatctt

SEQ ID NO: 13 - DNA, 20 bp, Artificial Sequence
  Cytochrome C antisense primer
ggtctgccctttctcccttc

SEQ ID NO: 14 - DNA, 20 bp, Artificial Sequence
  Caspase 3 sense primer
gagcttggaacggtacgcta

SEQ ID NO: 15 - DNA, 20 bp, Artificial Sequence
  Caspase 3 antisense primer
ccgtaccagagcgagatgac

SEQ ID NO: 16 - DNA, 21 bp, Artificial Sequence
  Caspase 8 sense primer
aacattcggaggcatttctgt

SEQ ID NO: 17 - DNA, 21 bp, Artificial Sequence
  Caspase 8 antisense primer
agaagagctgtaacctgtggc

SEQ ID NO: 18 - DNA, 20 bp, Artificial Sequence
  Caspase 9 sense primer
ctctgaagacctgcagtccc

SEQ ID NO: 19 - DNA, 20 bp, Artificial Sequence
  Caspase 9 antisense primer
ctgctccacattgccctaca

SEQ ID NO: 20 - DNA, 20 bp, Artificial Sequence
  P53 sense primer
accagggcaactatggcttc

SEQ ID NO: 21 - DNA, 20 bp, Artificial Sequence
  P53 antisense primer
agtggatcctggggattgtg

SEQ ID NO: 22 - DNA, 20 bp, Artificial Sequence
  BAD sense primer
cagcgtacgcacacctatcc

SEQ ID NO: 23 - DNA, 20 bp, Artificial Sequence
  BADantisense primer
cgggatcggacttcctcaag

SEQ ID NO: 24 - DNA, 20 bp, Artificial Sequence
  BID sense primer
tctgaggtcagcaacggttc

SEQ ID NO: 25 - DNA, 20 bp, Artificial Sequence
  BID antisense primer
tttgtcttcctccgacaggc

SEQ ID NO: 26 - DNA, 20 bp, Artificial Sequence
  BAX sense primer
ctggatccaagaccagggtg

SEQ ID NO: 27 - DNA, 20 bp, Artificial Sequence
  BAX antisense primer
cctttccccttcccccattc

SEQ ID NO: 28 - DNA, 20 bp, Artificial Sequence
  BCL2 sense primer
agcatgcgacctctgtttga

SEQ ID NO: 29 - DNA, 20 bp, Artificial Sequence
  BCL2 antisense primer
gccacacgtttcttggcaat

SEQ ID NO: 30 - DNA, 20 bp, Artificial Sequence
  BCL-XL sense primer
ttgtacctgcttgctggtcg

SEQ ID NO: 31 - DNA, 20 bp, Artificial Sequence
  BCL-XL antisense primer
cccggttgctctgagacatt

SEQ ID NO: 32 - DNA, 15 bp, Artificial Sequence
  BAK sense primer
cctgggccaacacgc

SEQ ID NO: 33 - DNA, 21 bp, Artificial Sequence
  BAK antisense primer
ctgtgggctgaagctgttcta